(19) United States
(12) Patent Application Publication
(10) Pub. No.: US 2019/0374242 A1
(43) Pub. Date: Dec. 12, 2019

(54) MULTI-FUNCTIONAL LAPAROSCOPIC SURGICAL APPARATUSES AND APPLICATIONS THEREOF

(71) Applicant: KATYA SURGICAL LTD, Tel-Aviv (IL)

(72) Inventors: Galit AVIGAL, Tel-Aviv (IL); Igor IGOV, Ramla (IL)

(73) Assignee: KATYA SURGICAL LTD, Tel-Aviv (IL)

(21) Appl. No.: 15/759,387

(22) PCT Filed: Oct. 28, 2016
(86) PCT No.: PCT/IB16/56523

§ 371 Date: Mar. 12, 2018

Related U.S. Application Data
(60) Provisional application No. 62/217,349, filed on Sep. 11, 2015.
(60) Provisional application No. 62/359,737, filed on Jul. 8, 2016.

(51) Int. Cl. A61B 17/295; A61B 17/32; A61B 17/3201; A61B 18/14; A61B 34/30

(52) U.S. Cl. CPC A61B 17/295; A61B 17/320016; A61B 17/00234; A61B 18/1445; A61B 34/30; A61B 17/3201

(57) ABSTRACT

Multi-functional laparoscopic surgical apparatuses (devices, systems) and applications (methods) for performing interventions in a subject's body. Exemplary embodiments are suitable for performing multiple different types of minimally invasive surgical procedures, such as tissue grasping, cutting, dissecting, fastening, and ligating, for example, via the same device. Exemplary apparatuses includes a tissue affecting device head that is reversibly convertible (transformable) between different tissue affecting configuations and modes (states) of operation, involving, for example, tissue dissecting, scissoring, and tissue grasping. Exemplary apparatuses and methods are implemented using a single, optionally, robotic, control unit. Also disclosed are exemplary embodiments of a surgical device including a longitudinal shaft enclosing a lumen having a cross section with a curved boundary, and a plurality of surgical instruments provided in the lumen, wherein at least two of the surgical instruments are packed according to a nested or/and concentric arrangement relative to the lumen curved boundary.

### FIELD OF THE INVENTION

The present invention, in some embodiments thereof, relates to laparoscopy, and more particularly, but not exclusively, to multi-functional laparoscopic surgical apparatuses and applications thereof, for performing surgical interventions in a body of a subject. Exemplary embodiments of the invention relate to laparoscopic surgical apparatuses (devices and systems) and methods that are suitable for performing multiple different types of minimally invasive surgical procedures, such as tissue grasping, cutting, dissecting, fastening, and ligating, among other possible surgical procedures.

### BACKGROUND OF THE INVENTION

In recent years, the use of laparoscopic instruments to perform minimally invasive surgical procedures on subjects has become increasingly popular. In such procedures, surgical instrumentation is introduced into the body of a subject in a minimally invasive approach via separate key-holes, and a laparoscope is inserted in a similar fashion for providing direct visualization of the surgical procedure. These surgical instruments are used according to the type of surgical intervention and may include any combination of grasper, scissors, dissectors, tissue fasteners, coagulating and ligating appliers, forceps, and the like. Usually, a surgeon operates different instruments using both hands. Typically, in such surgeries, the surgeon operates a grasper in one hand while using the other for operating a tissue effecting instrument. A very common combination of tissue affecting instrumentation required in a single laparoscopic operation includes a dissector, a tissue cutting instrument (usually a scissors), and a ligating electrode (usually in a form of hook). Introduction into the body of those diverse instruments is often made through several incisions or by frequently replacing instruments through a single incision.

Exemplary teachings in the field and art of the invention are provided in the following disclosures: U.S. Pat. Nos. 6,773,434; 5,984,938; and 5,219,354.

In spite of these and other teachings in the field and art of the invention, there is need for developing and practicing new or/and improved techniques (apparatuses and methods) for performing laparoscopic type surgical interventions in a body of a subject.

### SUMMARY OF THE INVENTION

The present invention, in some embodiments thereof, relates to laparoscopy, and more particularly, but not exclusively, to multi-functional laparoscopic surgical apparatuses and applications thereof, for performing interventions in a body of a subject. Exemplary embodiments of the invention relate to laparoscopic surgical apparatuses (devices and systems) and methods that are suitable for performing multiple different types of minimally invasive surgical procedures, such as tissue grasping, cutting, dissecting, fastening, and ligating, among other possible surgical procedures.

In exemplary embodiments, the laparoscopic surgical apparatuses and methods are employed for performing several different functions via the same device. In exemplary embodiments, the laparoscopic surgical devices and systems are reversibly convertible (transformable) between different tissue affecting configuations and modes (states) of operation, involving, for example, at least one of: tissue dissecting, tissue scissoring, and tissue grasping. In exemplary embodiments, the disclosed laparoscopic surgical apparatuses and methods are used and implemented for performing multiple different functions via a single, optionally, robotic, control unit (control mechanism).

Also disclosed are exemplary embodiments of a surgical device including a longitudinal shaft enclosing a lumen having a cross section with a curved boundary, and a plurality of surgical instruments provided in the lumen, wherein at least two of the surgical instruments are packed according to a nested or/and concentric arrangement relative to the lumen curved boundary.

According to an aspect of some embodiments of the present invention, there is provided a surgical device for performing surgical interventions in a body of a subject, the device comprising: a tissue affecting device head extending from distal end of a longitudinal shaft and including a first dissector jaw connected to a second dissector jaw with a pivot, each of the dissector jaws includes a slot extending therealong, a first scissors blade sized and configured for sliding, and locking into a first slot in the first dissector jaw, and a second scissors blade sized and configured for sliding, and locking into a second slot in the second dissector jaw; a jaws manipulator, configured for manipulating the first and second dissector jaws; and a control mechanism operatively connected, with the jaws manipulator, to the tissue affecting device head, and configured to selectively shift the dissector jaws from a closed state into an opened state.

In some embodiments of the surgical device, the tissue affecting device head is reversibly convertible between a tissue dissecting configuration, whereby the tissue affecting device head is structurally and functionally configured for dissecting soft tissue, and a tissue scissoring configuration, whereby the tissue affecting device head is structurally and functionally configured for scissoring soft tissue.

In some embodiments of the surgical device, the pivot, the control mechanism, and the jaws manipulator are operatively connected to the tissue affecting device head for facilitating operation of, and the reversible convertability between, the tissue dissecting configuration and the tissue scissoring configuration, whereby actuation force is transferrable to the dissector jaws and the tissue scissoring configuration, and whereby actuation force is also transferrable to the scissors blades via the dissector jaws.

According to some embodiments of the invention, in the tissue dissecting configuration the scissors blades are disposed within the longitudinal shaft, and in the tissue scissoring configuration the scissors blades are locked within the first and second dissector jaws.

According to some embodiments of the invention, the surgical device further comprises a pivot shifting mechanism configured to selectively shift the pivot relative to a cam surface, from a first pivot travel range that allows contact between the first and second dissector jaws to a second pivot travel range, thereby facilitating spacing between the first and second dissector jaws.

According to some embodiments of the invention, the pivot shifting mechanism includes a device configuration selector configured for the spacing between the first and second dissector jaws, so as to provide space for the scissors blades to slide therein, and for driving the scissors blades to slide and lock in the first and second slots.

According to some embodiments of the invention, the surgical device further comprises a scissors deploying mechanism operatively connected with the control mechanism and including a blades pusher member connected with a distal end thereof to each one of the scissors blades, and configured for pushing and pulling each one of the scissors blades along a path.

According to some embodiments of the invention, the first dissector jaw is curved with the first slot extending and curved therealong and the second dissector jaw is curved with the second slot extending and curved therealong.

According to some embodiments of the invention, the control mechanism includes a handle disposed at proximal end of the longitudinal shaft, and is configured for selectively actuating the jaws manipulator. According to some embodiments of the invention, the handle includes a lever arm. According to some embodiments of the invention, the control mechanism is positioned at proximal end of the longitudinal shaft, and includes a robotic arm configured for selectively actuating the jaws manipulator.

According to some embodiments of the invention, the scissors blades are connected to each other with a flexible link. According to some embodiments of the invention, the scissors blades are formed as a shear-like insert.

According to some embodiments of the invention, the surgical device is connectable to or integratable into a system for performing surgical interventions in a body of a subject, the system further comprising: a tissue grasping apparatus including a hollow grasping head having pivotally connected first and second grasping jaws configured for grasping tissue; a tissue grasping manipulator configured for forcibly manipulating the first and second grasping jaws from an opened state to a closed state, such that the hollow grasping head in the closed state forms a grasping head lumen sized and shaped to allow the tissue affecting device head to pass therethrough and therealong; and a shifting mechanism configured for shifting the surgical device to an operative position within, or distal to, the hollow grasping head.

According to an aspect of some embodiments of the present invention, there is provided a system for performing surgical interventions in a body of a subject, the system comprising: a surgical device including a longitudinal shaft upon which is mounted a tissue affecting device head; a tissue grasping apparatus including a hollow grasping head having pivotally connected first and second grasping jaws configured for grasping tissue; a tissue grasping manipulator configured for forcibly manipulating the first and second grasping jaws from an opened state to a closed state, such that the hollow grasping head in the closed state forms a grasping head lumen sized and shaped to allow the tissue affecting device head to pass therethrough and therealong; and a shifting mechanism configured for shifting the surgical device to an operative position within, or distal to, the hollow grasping head.

According to some embodiments of the invention, the surgical device includes a dissector head equipped with a set of first and second dissector jaws. According to some embodiments of the invention, the surgical device includes a scissors head equipped with a set of first and second scissor jaws.

According to some embodiments of the invention, each of the first and second grasping jaws is configured as a half-tube with a toothed edge. According to some embodiments of the invention, the tissue affecting device head is reversibly convertible between a tissue dissecting configuration and a tissue scissoring configuration.

According to some embodiments of the invention, the grasping head lumen is sized for housing the tissue affecting device head therein, thereby allowing combined and unified operation of the grasping head and the tissue affecting device head as an integral grasper head.

According to some embodiments of the invention, each of the first and second grasping jaws is at least partially filled with viscoelastic material or/and malleable material, configured to at least partially conform to a corresponding dissector jaw submerging therein, thereby facilitating formation of contact area for grasping, or/and uniform distribution of compression stresses, when applying the integral grasper head for grasping soft tissue.

According to some embodiments of the invention, each of the first and second grasping jaws includes a sloped surface shaped and dimensioned such that the tissue affecting device head, when engaging and pushing against the sloped surface, forces the grasping jaws to radially retreat so as to allow the tissue tissue affecting device head to advance across distal end of the grasping head. According to some embodiments of the invention, the grasping apparatus includes an outer tube configured for connecting between the tissue grasping manipulator and the grasping head.

According to some embodiments of the invention, the system further comprises a cautery electrode connected with cautery means, the cautery electrode is extendable from a recess in the grasping head. According to some embodiments of the invention, the cautery electrode is configured in a form of a hook. According to some embodiments of the invention, the cautery electrode is configured for being forced to revolve from a horizontal position to a vertical position when extending from the recess to a fully deployed position.

According to an aspect of some embodiments of the present invention, there is provided a method for performing surgical interventions in a body of a subject, the method comprising:

introducing, through an incision in the body, a surgical device comprising a tissue affecting device head extending from a distal end of a longitudinal shaft and having pivotally connected first and second dissector jaws, and further comprising a first scissors blade and a second scissors blade each sized and configured for sliding, and locking within a corresponding one of the first and second dissector jaws, wherein the tissue affecting device head is reversibly convertible between a tissue dissecting configuration and a tissue scissoring configuration, and between an opened state and a closed state via a control mechanism extending from proximal end of the longitudinal shaft; dissecting tissue using the surgical device when the tissue affecting device head is in the tissue dissecting configuration, and, by opening the tissue affecting device head and closing the tissue affecting device head around the tissue; converting the tissue affecting device head into the tissue scissoring configuration by the sliding and locking the first and second scissors blades within the first and second dissector jaws; and cutting the tissue by opening the tissue affecting device head and closing the tissue affecting device head around the tissue.

According to some embodiments of the invention, the method further comprises introducing a tissue grasping apparatus via the incision, wherein the tissue grasping apparatus includes an outer tube sized to accommodate and facilitate sliding therethrough the longitudinal shaft and the tissue affecting device head, wherein the outer tube is connected via a distal end thereof to a hollow grasping head having pivotally connected first and second grasping jaws, and the grasping head is forcibly transformable between an opened state and a closed state.

According to some embodiments of the invention, the method comprises sliding the surgical device through the tissue grasping apparatus. According to some embodiments of the invention, the method comprises grasping the tissue by opening the grasping head and closing the grasping head around the tissue. According to some embodiments of the invention, the method further comprises deploying a cautery electrode disposed within the outer tube, thereby facilitating at least one of: tissue cutting, tissue coagulation, or tissue dessication.

According to an aspect of some embodiments of the present invention, there is provided a surgical device comprising: a longitudinal shaft enclosing a lumen having a cross section with a curved boundary; and a plurality of surgical instruments provided in the lumen, wherein at least two of the surgical instruments are packed according to a nested or/and concentric arrangement relative to the lumen curved boundary.

According to some embodiments of the invention, each of the plurality of surgical instruments is configured for being selectively, slidably protrudable from remainder of the surgical instruments, into a deployable form, thereby facilitating use of the deployable form for performing a single surgical procedure (maneuver), or a set of two or more surgical procedures (maneuvers). According to some embodiments of the invention, the deployable form is configured for performing a plurality of different types of single surgical procedures (maneuvers), or a plurality of different types of sets of two or more surgical procedures (maneuvers).

All technical or/and scientific words, terms, or/and phrases, used herein have the same or similar meaning as commonly understood by one of ordinary skill in the art to which the invention pertains, unless otherwise specifically defined or stated herein. Exemplary embodiments of methods (steps, procedures), apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict, the patent specification, including definitions, will control.

Implementation of some embodiments of the invention can involve performing or completing selected tasks manually, automatically, or a combination thereof. Moreover, according to actual instrumentation and equipment of some embodiments of the invention, several selected tasks could be implemented by hardware, by software, by firmware, or a combination thereof, using an operating system.

For example, hardware for performing selected tasks according to embodiments of the invention could be implemented as a chip, as a circuit, or a combination thereof. As software, selected tasks of some embodiments of the invention could be implemented as a plurality of software instructions being executed by a computer using any suitable operating system. In an exemplary embodiment of the invention, one or more tasks of exemplary embodiments of the method or/and system as described herein are performed by a data processor, such as a computing platform for executing a plurality of instructions. Optionally, the data processor includes a volatile memory for storing instructions or/and data. Alternatively or additionally, optionally, the data processor includes a non-volatile storage, for example, a magnetic hard-disk or/and removable media, for storing instructions or/and data. Optionally, a network connection is provided as well. Optionally, a display or/and a user input device such as a keyboard or mouse is provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D are simplified schematics side views of an exemplary device for performing surgical interventions in a body of a subject, the device is transformable between a tissue dissecting configuration (FIGS. 1A-1B), and a tissue scissoring configuration (FIGS. 1C-1D) and between an opened state (FIGS. 1B and 1D), and a closed s state (FIGS. 1A and 1C), in accordance with some embodiments of the invention;

FIGS. 2A-2C are perspective views showing an exemplary device of the invention, wherein the device is in a tissue dissecting configuration and in a partially opened state (FIG. 2A), in an intermediate configuration wherein the jaws are spaced apart and parallel to each other (FIG. 2B), and in a tissue scissoring configuration when in a closed state (FIG. 2C), in accordance with some embodiments of the invention;

FIGS. 3A-3B demonstrate top respective views of the exemplary device of FIGS. 2A-2C, wherein a curved slot extends therealong a longitudinal axis of a dissector jaw (FIG. 3A) and wherein scissors blades are curved and locked within the curved slot (FIG. 3B), in accordance with some embodiments of the invention;

FIGS. 4A-4E show other views of the exemplary device of FIGS. 2A-2C, including a side cut view of the exemplary device (FIG. 4A), and perspective views of separated components thereof: jaws manipulator (FIG. 4B), levers retracting manipulator (FIG. 4C), levers compressing manipulator (FIG. 4D), and blades pusher head (FIG. 4E), in accordance with some embodiments of the invention;

FIGS. 5A-5B show a side cut view of the exemplary device of FIGS. 2A-2C in a tissue dissecting configuration when in closed state (FIG. 5A) and a tissue dissecting configuration when in the opened state (FIG. 5B), in accordance with some embodiments of the invention;

FIGS. 6A-6D are side cut views demonstrating pivot shifting mechanism, provided in the exemplary device of FIGS. 2A-2C, of pivot in a first range (FIGS. 6A-6B), and pivot in a second range (FIGS. 6C-6D), in accordance with some embodiments of the invention;

FIGS. 7A-7C are perspective views demonstrating exemplary blades pusher member (FIG. 7A), and exemplary scissors head, when in the opened state (FIG. 7B), and when in the closed state (FIG. 8C), in accordance with some embodiments of the invention;

FIGS. 8A-8B are perspective views of an exemplary device of the invention, wherein the device is in a tissue scissoring configuration, when in an opened state (FIG. 8A) and in the tissue dissecting configuration when in the closed state (FIG. 8B), in accordance with some embodiments of the invention;

FIGS. 9A-9C show a simplified cross section view (FIG. 9A) and respective views (FIGS. 9B-9C) of a surgical system of the invention, wherein the system includes a tissue grasping apparatus, in accordance with some embodiments of the invention;

FIGS. 10A-10B are respective views of the system of the invention, wherein an outer tube is retracted and wherein the system is in the tissue dissecting configuration (FIG. 10A) and in the tissue scissoring configuration (FIG. 10B), in accordance with some embodiments of the invention;

FIGS. 11A-11B are respective views of the system of the invention, wherein the tissue grasping head and the dissector head are integrally operated (FIG. 11A), or wherein the tissue grasping head includes a viscoelastic or partially filled malleable component (FIG. 11B), in accordance with some embodiments of the invention;

FIGS. 12A-12C are respective views of an outer tube and a tissue grasping head, wherein at least one of the grasping jaws includes a sloped surface allowing grasping jaws to retreat radially when dissector head pushes against the sloped surface, in accordance with some embodiments of the invention;

FIGS. 13A-13B are respective views of an outer tube that includes a cautery electrode positioned within a recess in the grasping head; in accordance with some embodiments of the invention;

FIG. 14 is a flow chart illustrating an exemplary method of performing a surgical intervention in a body of a subject utilizing the surgical device of the invention, in accordance with some embodiments of the present invention;

FIG. 15 is a flow chart illustrating an exemplary method of performing a surgical intervention in a body of a subject utilizing the surgical system of the invention, in accordance with some embodiments of the present invention;

FIGS. 16A-16C are respective views of an exemplary device of the invention, wherein the dissector head transforms to a cutting head by integrating with a side blade, in accordance with some embodiments of the invention; and

FIGS. 17A-17F are respective views of an exemplary device of the invention including a plurality of surgical instruments packed in a nested or/and concentric arrangement in a lumen of a longitudinal shaft, each surgical instrument is selectively slidably protrudable to the rest into a deployable form, in accordance with some embodiments of the invention.

### DETAILED DESCRIPTION OF SPECIFIC EMBODIMENTS OF THE INVENTION

The present invention relates to multi-functional laparoscopic surgical apparatuses and applications thereof, for performing interventions in a body of a subject. Exemplary embodiments of the invention relate to laparoscopic surgical apparatuses (devices and systems) and methods that are suitable for performing multiple different types of minimally invasive surgical procedures, such as tissue grasping, cutting, dissecting, fastening, and ligating, among other possible surgical procedures.

In exemplary embodiments, the laparoscopic surgical apparatuses and methods are employed for performing several different functions via the same device. In exemplary embodiments, the laparoscopic surgical devices and systems are reversibly convertible (transformable) between different tissue affecting configuations and modes (states) of operation, involving, for example, at least one of: tissue dissecting, tissue scissoring, and tissue grasping. In exemplary embodiments, the disclosed laparoscopic surgical apparatuses and methods are used and implemented for performing multiple different functions via a single, optionally, robotic, control unit (control mechanism).

Also disclosed are exemplary embodiments of a surgical device including a longitudinal shaft enclosing a lumen having a cross section with a curved boundary, and a plurality of surgical instruments provided in the lumen, wherein at least two of the surgical instruments are packed according to a nested or/and concentric arrangement relative to the lumen curved boundary.

Currently known and used laparoscopic surgical devices may be limited in function or/and operability, for example, with respect to at least one limitation of being operably controlled via separate control units, thereby rendering actuation and manipulation of such devices complicated and time consuming. In view of the current state of the art, including at least some of the just stated limitations, there is need for developing and practicing improved or/and new techniques (apparatuses and methods) for performing laparoscopic type surgical interventions in a body of a subject.

The disclosed laparoscopic surgical apparatuses (devices and systems) are multi-functional and can be readily actuated. Implementation of exemplary embodiments of the invention afford minimizing the number of incisions typically needed for performing a particular endoscopic procedure, or/and the number of different devices required in endoscopic procedures. By implementing exemplary embodiments of the invention, a tissue in the body of a subject may be manipulated to facilitate different types of tissue affecting actions, such as, tissue grasping or/and tissue dissecting or/and tissue cutting. Exemplary embodiments of the invention may be implemented remotely by the user via a single and same control unit.

The term ‘tissue affecting’, as used herein, in a non-limiting manner, for example, in the context of a device or component thereof having the capability of ‘affecting tissue’ in the body of a subject, refers to any act, action, or process that ‘affects tissue’. Exemplary ‘tissue affecting’ acts, actions, or processes, are ‘grasping’ tissue, ‘dissecting’ tissue', ‘cutting’ tissue, ‘tearing’ tissue, ‘shearing’ tissue, and ‘scissoring’ tissue.

The term ‘grasp’, as used herein, in a non-limiting manner, for example, in the context of the act, action, or process of ‘grasping’, refers to pressing (tightly or otherwise), usually temporarily, against a target tissue (normally, soft tissue, but can also be other types of tissue), using opposing contact surfaces that are forced against each other. In the art, a surgical grasper is commonly known as a surgical device having two opposing jaws, optionally, each jaw having a large contact surface relative to its size. A surgical grasper has applications in different surgical or/and endoscopic procedures for grasping/clamping of tissue and small tubular structures.

The term ‘dissect’, as used herein, in a non-limiting manner, for example, in the context of the act, action, or process of ‘dissecting’, refers to opening up soft tissue, typically, involving cutting of the soft tissue. In common surgical or/and laparoscopic procedures, soft tissue dissection involves increasing the size of a tear, incision, or any opening in the tissue, by affecting the tissue in order to enlarge the dimension (e.g., length or diameter) of the opened tissue. Dissection may be applied by mechanical means (i.e., by applying mechanical force), commonly by applying blunt means, or/and by directing energy (e.g., heat). A surgical tissue dissector is particularly designed and structured to at least be configured for soft tissue dissection. Commonly, and as opposed to surgical graspers, for example, a typical surgical dissector used in laparoscopy procedures includes two opposing jaws having specific design allowing it to effectively enter through tissue layers or/and openings and to manipulate the tissue prior to, during, or/and after, dissection thereof. The act, action, or processs, of ‘dissecting’ typically involves the act, action, or process, of ‘cutting’ or ‘tearing’, as defined below.

The term ‘cut’, as used herein, in a non-limiting manner, for example, in the context of the act, action, or process of ‘cutting’, refers to separating tissue between portions thereof by applying a sharp edge, or focused energy, thereupon such that formerly unified tissue portions, layers, or types, become disconnected and separated. Cutting usually includes penetrating through tissue thickness along a chosen line of separation (cutting line).

The term ‘tear’, as used herein, in a non-limiting manner, for example, in the context of the act, action, or process of ‘tearing’, refers to forcing tissue portions, layers, or types, to become disconnected (separated) by applying opposing forces (e.g., by pulling or pushing). Tissue tearing can be affected by either blunt or sharp instruments.

The term ‘shear’, as used herein, in a non-limiting manner, for example, in the context of the act, action, or process of ‘shearing’, refers to a deformation (e.g., possibly also cutting or tearing) of an object or material in which parallel planes remain parallel but are shifted in a direction parallel to themselves. A tissue affecting device may combine different types of tissue affecting means, such as a scalpel that cuts tissue by way of combining shearing and tearing of different tissue layers.

The term ‘scissor’, as used herein, in a non-limiting manner, for example, in the context of the act, action, or process of ‘scissoring’, as used herein, in a non-limiting manner, refers to cutting or shearing via relative revolving or pivoting of one (scissor) blade next to another, such as with two crossed pivoting (scissor) blades as found in most scissors. By contrast, the act, action, or procedure of ‘chopping’ (e.g., by way of applying a guillotine) may involve use of a single blade moving in a straight/planar direction in order to cut an object or material against a support (which may include a blade).

The term ‘convertible’, as used herein, in a non-limiting manner, for example, in the context of a device or component thereof having the characteristic or property of being ‘convertible’, refers to an entity or object, such as a device or component thereof, that is changeable or transformable from a first type of (structural or/and functional) configuration to a second type of (structural or/and functional) configuration.

The phrase ‘reversibly convertible’, as used herein, in a non-limiting manner, for example, in the context of a device or component thereof having the characteristic or property of being ‘reversibly convertible’, refers to an entity or object, such as a device or component thereof, that is reversibly changeable or transformable between a first type of (structural or/and functional) configuration and a second type of (structural or/and functional) configuration. In such context, the second type of (structural or/and functional) configuration is ‘convertible’ back to the first type of (structural or/and functional) configuration.

In exemplary embodiments of the herein disclosed invention, the system for performing surgical interventions in a body of a subject includes a surgical device that has a ‘reversibly convertible’ tissue affecting device head, namely, a tissue affecting device head that is reversibly changeable or transformable between a first type of (structural and functional) configuration, being a ‘dissecting’ configuration, and a second type of (structural and functional) configuration, being a ‘scissoring’ configuration. In exemplary embodiments, the second type of (structural and functional) configuration, namely, the ‘scissoring’ configuration, is ‘convertible’ back to the first type of (structural and functional) configuration, namely, the ‘dissecting’ configuration.

Some embodiments of the invention relate to devices that are (reversibly) transformable (changeable) between a dissecting configuration and a scissoring configuration and vice versa.

According to some embodiments, the device includes a tissue affecting device head (e.g., in a form of a dissector head) including pivotally connected dissector jaws, and an elongated shaft attached to a proximal end of the dissector head. According to some embodiment, the device further includes scissors blades confined within the shaft and deployable upon command within the dissectors jaws. According to some embodiments, the tissue affecting device head is reversibly convertible into a configuration of a scissors head including pivotally connected scissors blades and an elongated shaft attached to a proximal end of the scissors head. According to some embodiment, the device further includes dissector jaws confined within the shaft and deployable upon command within the scissors blades.

According to some embodiments, the device includes a control unit (control mechanism) for selectively operating the systems' various configurations. According to some embodiments, the control unit selectively or/and controllably transforms the device from a tissue scissoring configuration to a tissue dissecting configuration and vice versa. According to some embodiments, the control unit selectively or/and controllably shifts the device from a closed state to an opened state and vice versa. According to some embodiments, opening and closing of the device head, either in its scissoring configuration or its dissecting configuration, is effected by a jaw manipulator disposed within the shaft and operably connected to the control unit and the device head (jaws).

According to some embodiments, the device further includes a tissue grasping apparatus configured for grasping tissue or/and for allowing passage therethrough of the surgical device. According to some embodiments, the tissue grasping apparatus is connected with the surgical device or is provided as a separate unit. According to some embodiments, the tissue grasping apparatus and the surgical device are both controlled via the same control unit. According to some embodiments, the tissue grasping apparatus and the surgical device are controlled by separate control units.

According to some embodiments, the present invention further provides a system including a surgical device transformable between a tissue dissecting configuration or/and a tissue scissoring configuration or/and a tissue grasping configuration.

Also disclosed herein are exemplary embodiments of a surgical device including a longitudinal shaft enclosing a lumen having a cross section with a curved boundary, and a plurality of surgical instruments provided in the lumen, wherein at least two of the surgical instruments are packed according to a nested or/and concentric arrangement relative to the lumen curved boundary.

According to an aspect of some embodiments of the present invention, there is provided a surgical device for performing surgical interventions in a body of a subject. In exemplary embodiments, the device includes: a tissue affecting device head extending from distal end of a longitudinal shaft and including a first dissector jaw connected to a second dissector jaw with a pivot, each of the dissector jaws includes a slot extending therealong, a first scissors blade sized and configured for sliding, and locking into a first slot in the first dissector jaw, and a second scissors blade sized and configured for sliding, and locking into a second slot in the second dissector jaw. In exemplary embodiments, the surgical device further includes a jaws manipulator, configured for manipulating the first and second dissector jaws. In exemplary embodiment, the surgical device further includes a control mechanism operatively connected, with the jaws manipulator, to the tissue affecting device head, and configured to selectively shift the dissector jaws from a closed state into an opened state.

In exemplary embodiments, the tissue affecting device head is reversibly convertible between a tissue dissecting configuration, whereby the tissue affecting device head is structurally and functionally configured for dissecting soft tissue, and a tissue scissoring configuration, whereby the tissue affecting device head is structurally and functionally configured for scissoring soft tissue.

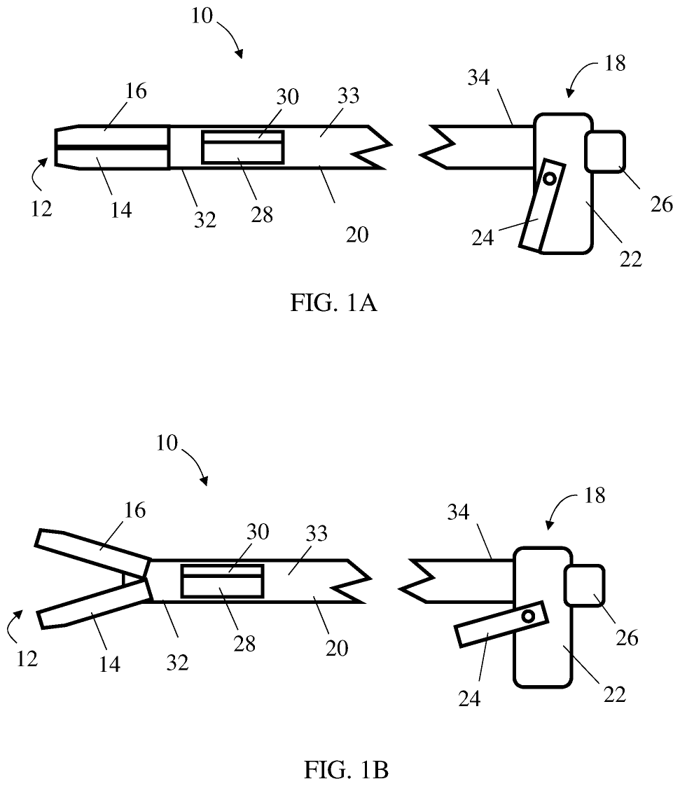

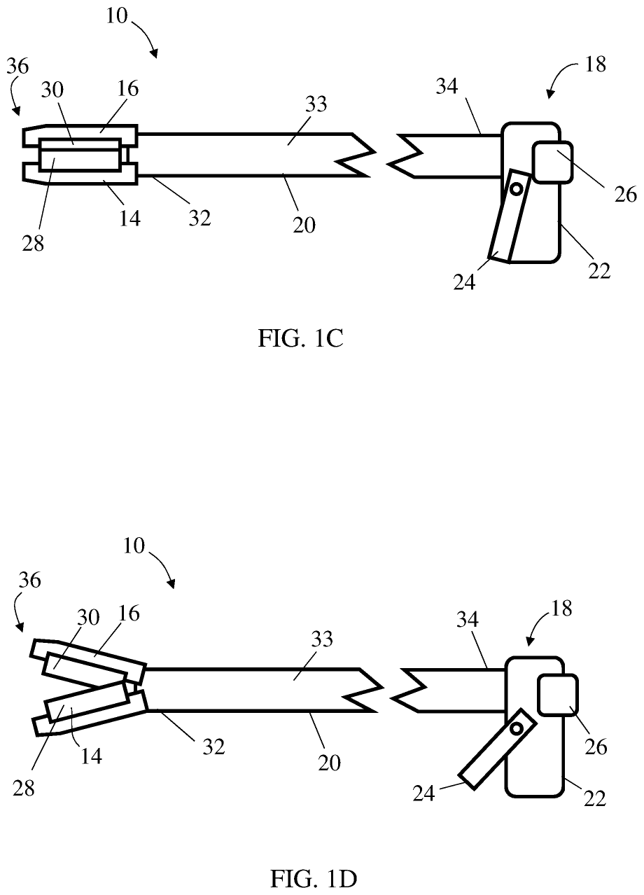

Reference is made now to FIGS. 1A-1D, which show an exemplary device 10 for performing surgical interventions in a body of a subject, in accordance with some embodiments of the invention. Device 10 includes a tissue affecting device head 12, including a first dissector jaw 14 and a second dissector jaw 16, that is transformable between a tissue dissecting configuration (FIGS. 1A-1B) and a tissue scissoring configuration (FIGS. 1C-1D). Device 10 further includes an elongated shaft 20 extending from a shaft proximal end 34 and a shaft distal end 32 and enclosing a lumen 33.

According to some embodiments, the device (and its device head 12) may be operated by a control unit, such as control mechanism 18. According to some embodiments, control mechanism 18 extends from the proximal end 34 of shaft 20.

The term ‘control unit’, as used herein, is interchangeable with the term ‘control mechanism’, and refers to a unit, mechanism, or component, that controls the reversible conversions or transformations of the herein disclosed exemplary devices between or among various configurations and modes (states) of operation, so as to facilitate the device to perform various tissue affecting functions and procedures.

Control mechanism 18 is operatively connected to tissue affecting device head 12. According to some embodiments, in a ‘closed state’, dissector jaws 14 and 16 are in contact or in close proximity and in an ‘opened state’ jaws 14 and 16 are spaced apart. In one embodiment, control mechanism 18 selectively and controllably transforms between a tissue dissecting configuration to a tissue scissoring configuration. In one embodiment, control mechanism 18 can be used to selectively and controllably transform device 10 between a tissue scissoring configuration and a tissue dissecting configuration. In one embodiment, control mechanism 18 can be applied to selectively and controllably shift dissector jaws 14 and 16 from a closed state, in both tissue dissecting configuration (FIG. 1A) and tissue scissoring configuration (FIG. 1C), into an opened state (FIGS. 1B and 1D, respectively), and vice versa.

According to some embodiments, control mechanism 18 is manually or/and remotely operated by a user, typically a surgeon. Control mechanism 18 may include a handle 22 through which the device 10 can be controlled. According to some alternative embodiments, control mechanism 18 includes or is in the form of a robotic arm and allows device 10 manipulation by the surgeon. Handle 22 may include elements for actuating and functioning device 10 configurations or/and states. In some embodiments, control mechanism 18 includes a lever arm 24 configured for selectively actuating tissue affecting device head 12. Optionally, additionally or alternatively, control mechanism 18 includes actuator 26 that is configured for selectively transforming the device between one type of tissue affecting to a different type of tissue affecting, such as between a tissue dissecting configuration and a tissue scissoring configuration.

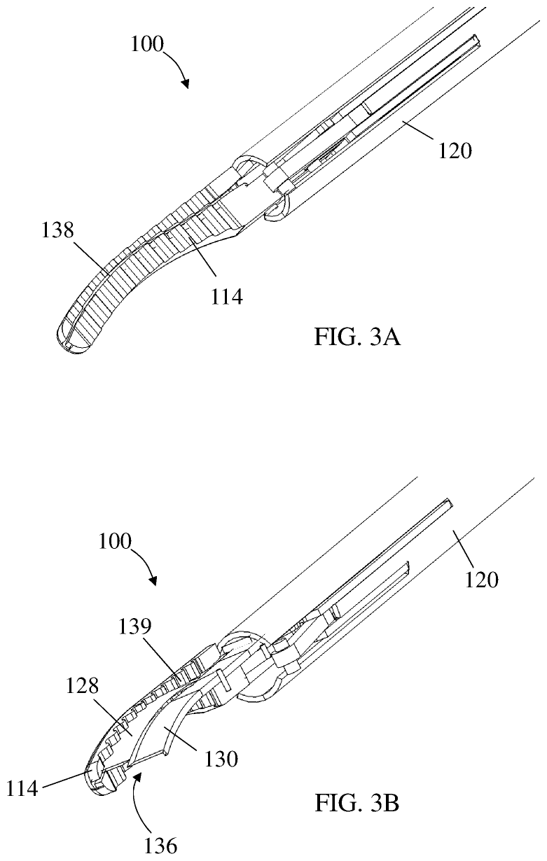

Some embodiments of the invention refer to the device of the invention that provides a dissector that may be selectively and controllably transformed (in both function and structure) to scissors, and optionally vice versa. In accordance with those embodiments, device 10 includes a first scissors blade 28 and a second scissors blade 30. First scissors blade 28 and second scissors blade 30 can be assembled into dedicated docks in the tissue affecting device head 12 to form a scissors head 36 (shown in FIGS. 1C-1D) being configured for cutting, such as by shearing, a soft tissue. Scissors blades 28 and 30 are movable between a retracted position, wherein the blades are disposed and confined within shaft 20 (shown in FIGS. 1A-1B) and a deployed configuration, wherein the blades are disposed between dissector jaw 14 and dissector jaw 16. Blades 28 and 30 may be disposed or/and docked within jaws 14 and 16, respectively, in any suitable way or form. In some exemplary embodiments, and as will be described in more details with reference to FIGS. 3A-3B, at least one of scissors blades 28 and 30 occupies a slot or a channel within a corresponding jaw 14 or/and 16, respectively, when device 10 is in the scissors configuration. In some embodiments, first and second scissors blades 28 and 30 are configured for sliding and curving within, and locking into, first jaw 14 and second jaw 16, respectively.

When the device is in the tissue dissecting configuration, tissue affecting device head 12 is operable to dissect a tissue, while scissors first and second blades 28 and 30, respectively, being disposed within shaft 20. Tissue affecting device head 12 is shaped, sized and configured for penetrating via relatively small tear, cut or incision in a soft tissue, and forcibly open to further tear or cut the tissue along a dissection line.

Tissue affecting device head 12 opening and closing is manually or/and remotely controlled by control mechanism 18. In some embodiments of the invention, control mechanism 18 is operatively connected with tissue affecting device head 12 such that manipulation of control mechanism 18, optionally via handle 22 effects opening and closing of tissue affecting device head 12. In some exemplary embodiments, tissue affecting device head 12 opening is effected via a lever arm 24 upward manipulation and tissue affecting device head 12 closing is effected via a lever arm 24 downward manipulation.

When the device is in the scissoring configuration, first and second scissors blades 28 and 30 are deployed and fixed within first and second jaws 14 and 16, respectively.

Transformation of the tissue affecting device head 12 into configuration of a scissors head 36, by deploying scissors blades 28 and 30, may be manually or/and remotely controlled by control mechanism 18, such as by operating actuator 26. Such transformation into the scissors head 36 configuration is effected in order to facilitate functional or/and structural performance associated with use of surgical scissors once blades are in place.

Transformation into the scissors configuration is accomplished by first rearranging relative positioning of jaws 14 and 16. In some such embodiments, operating actuator 26 effects spacing between dissector jaws 14 and 16, thereby creating room for scissors blades 28 and 30. Optionally, operating actuator 26 actuates a blades deploying mechanism 27 that effects the spacing between dissector jaws 14 and 16. Spacing between the jaws optionally includes a non-pivotal shift between jaws 14 and 16, or is optionally associated with a substantially parallel shifting between the jaws (as shown, for example, in FIG. 1C relative to FIG. 1A). Following adequate (e.g., predefined) spacing between jaws 14 and 16, scissors blades 28 and 30 are driven into sliding within jaws 14 and 16, respectively. At least one of the blades may curve to conform with optionally a curved slot in the respective jaw. Once fully sided within the respective slot, each blade is optionally locked in position.

According to some embodiments, scissors head 36 is operable to cut a tissue by opening and closing jaws 14 and 16 via same mechanism used for dissecting a tissue by tissue affecting device head 12. In accordance with those embodiments, scissors head 36 opening is effected via a lever arm 24 upward manipulation and scissors head 36 closing is effected via a lever arm 24 downward manipulation.

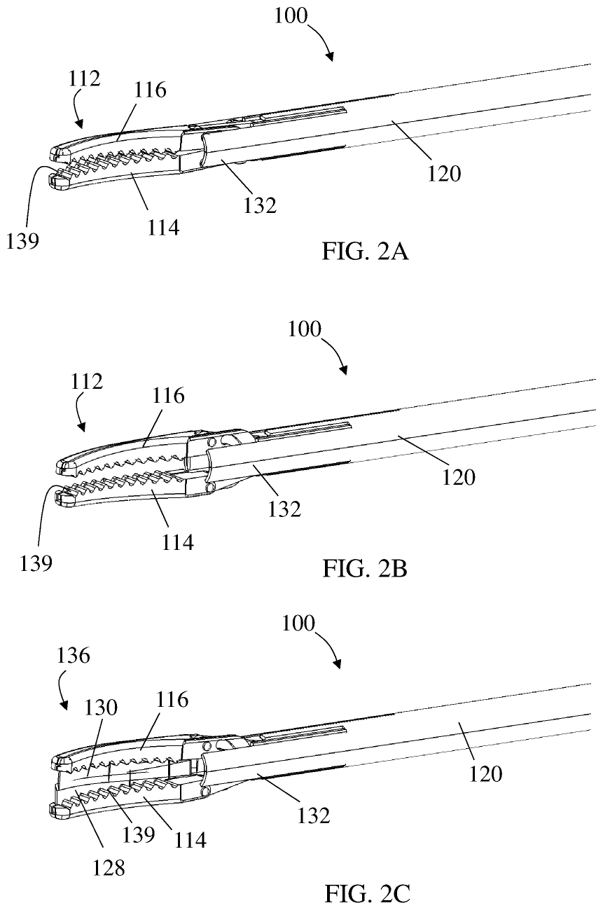

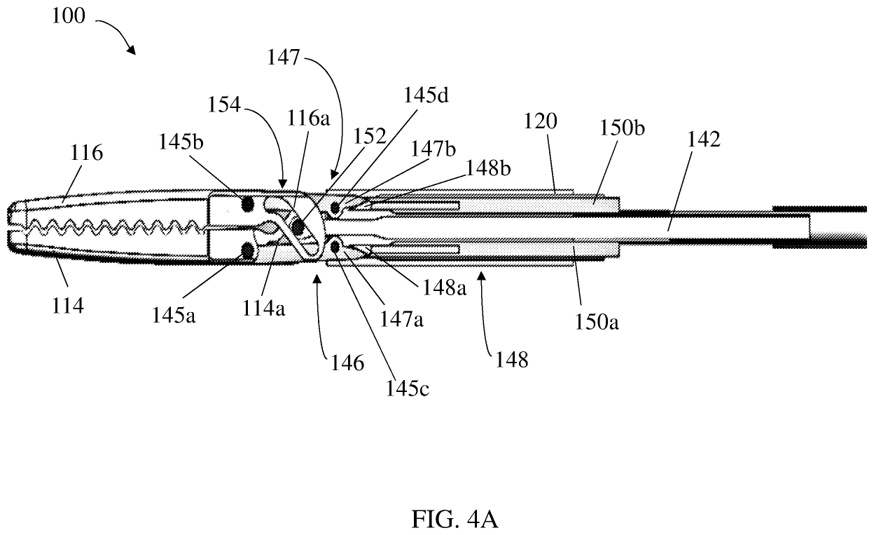

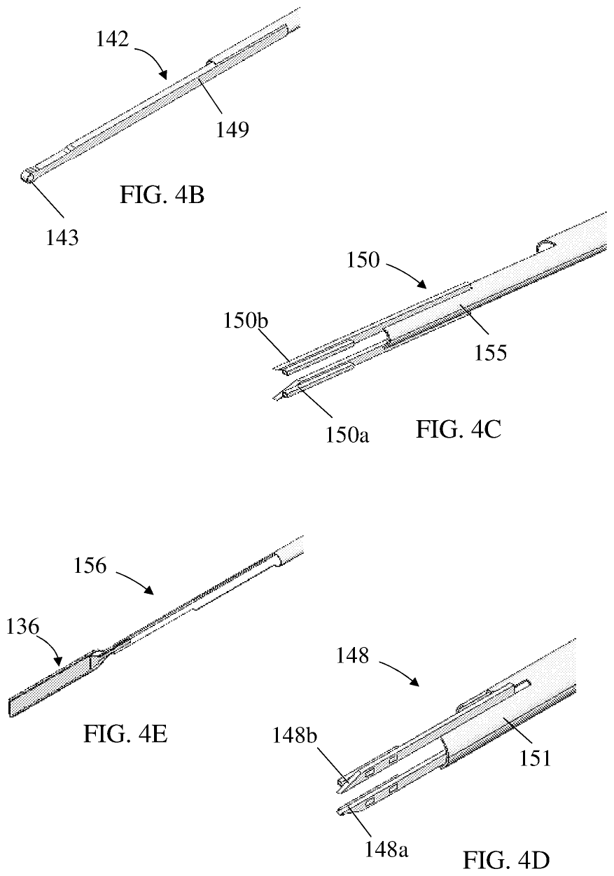

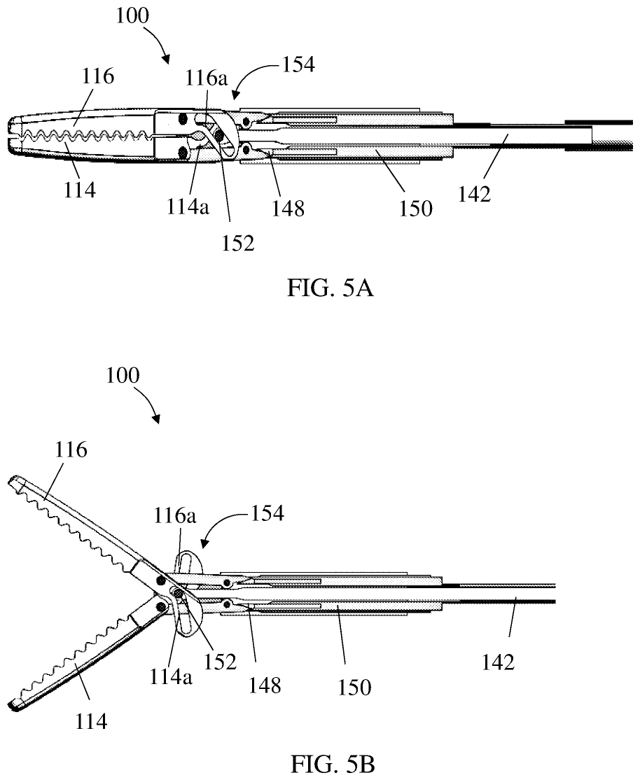

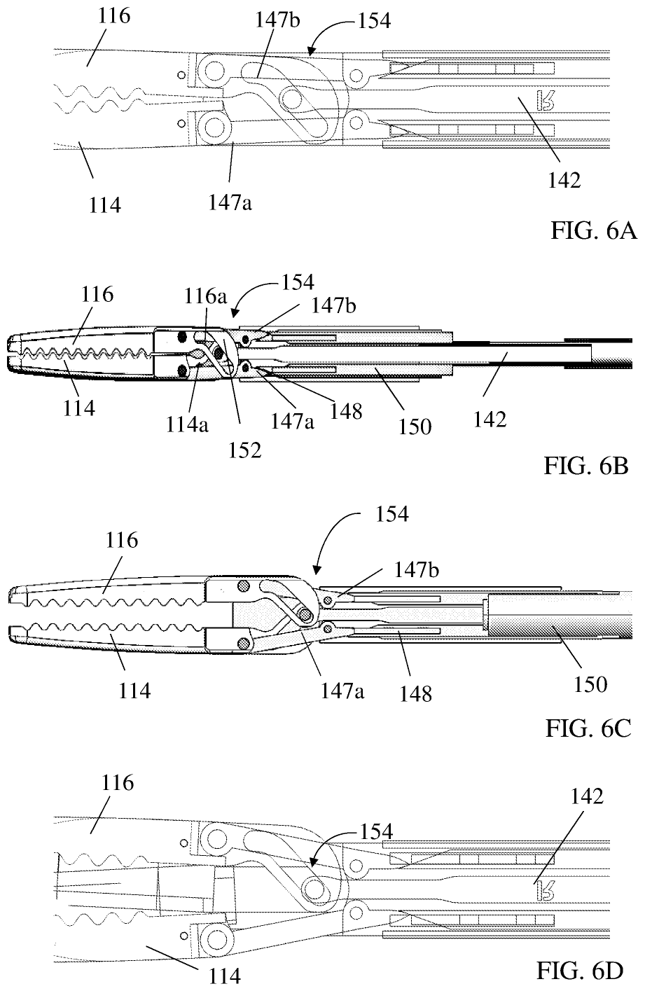

Reference is now made to FIGS. 2-6 showing functional and structural aspects of an exemplary device 100, in accordance with some embodiments of the invention. FIGS. 2A-2C show an exemplary distal portion of surgical device 100 including tissue affecting device head 112 coupled to a shaft distal end 132. FIGS. 3A-3B show respective partial views of device 100 and tissue affecting device head 112. FIGS. 4A-4D show a side cut view of device 100 (FIG. 4A), and respective isometric views of separated components thereof. FIGS. 5A-5B show a side cut view of an exemplary device of the invention in the tissue dissecting configuration when in the closed state (FIG. 5A) and the tissue dissecting configuration when in the opened state (FIG. 5B). FIGS. 6A-6D are side cut views demonstrating pivot shifting mechanism, of pivot in a first range (FIGS. 6A-6B), and pivot in a second range (FIGS. 6C-6D).

Referring to FIGS. 2A-2C, device 100 is shown in separate configurations: a tissue dissecting configuration (FIG. 2A), a tissue scissoring configuration (FIG. 2C), in which tissue affecting device head 112 transformed structurally and functionally into a scissors head 136, and an intermediate configuration (FIG. 2B) for facilitating these transformations.

Device 100 may be configured for use with a control mechanism (e.g., handheld or/and hand-operated) such as control mechanism 18 previously presented.

Tissue affecting device head 112 includes pivotally attached first dissector jaw 114 and second dissector jaw 116. Device 100 further includes scissors blades 128 and 130 provided within shaft 120 of device 100, when device 100 is in the tissue dissecting configuration, (FIG. 2A) and deployable, upon transforming into the tissue scissoring configuration (e.g., by a user actuation) to accommodate a space within first and second jaws 114 and 116, respectively (FIG. 2C).

At least one of jaws 114 and 116 optionally defines an internal surface 139 suitable for manipulating a tissue, such as by grasping, holding an instrument and the alike. Surface 139 may be toothed, jagged or may include parallel protrusions.

FIG. 2B shows device 100 in an intermediate configuration where jaws 114 and 116 are spaced so that blades 128 and 130 can be emerged from shaft 120 to slide along and occupy a space within jaws 114 and 116, respectively. Jaws 114 and 116 are spaced apart, upon actuation, optionally by a user, via a dedicated actuator (such as actuator 26 disposed within control mechanism 18).

FIG. 2C shows the device 100 in the scissoring configuration, in which blades 128 and 130 are disposed within jaws 114 and 116, respectively, thereby forming scissors head 136.

As shown in FIGS. 3A-3B, dissector jaw 114 includes a blade slot 138 extending and optionally curving therealong. Blade slot 138 is sized and shaped to allow scissors blade sliding therein or/and accommodating a scissors blade, or/and locking a scissors blade. According to some embodiments, first scissors blade 128 is sized and configured for sliding, curving and locking into a first blade slot 138 in first jaw 114. Although illustrated with respect to jaw 114, an oppositely positioned blade slot (which may be identical to blade slot 138) may be present within jaw 116. In accordance with this embodiment, second scissors blade 130 is sized and configured for sliding, curving and locking into the second blade slot (similar or identical to slot 138) in second jaw 116. According to some embodiments, jaws 114 and 116 are curved, and the corresponding blade slots are curved to comply with the curvature of jaws 114 and 116. Blade slots including blade slot 138 are provided in a slight lateral shift one to the other in a way that allows proper scissoring action between the blades, once deployed.

Blade slot 138 may extend along a longitudinal axis, optionally from a proximal end of dissector jaws or above, to a distal end or a position proximally spaced from the distal end of jaws 114 or/and 116. Scissors blades deployment and position within the jaws 114 and 116 afford scissors blades 128 and 130 assembling into scissors head 136 (as shown in FIGS. 2C and 3B). Scissors head 136 is configured for cutting, optionally by shearing, a soft tissue when positioned between scissors blades 128 and 130 upon closing tissue affecting device head 112.

As shown in FIG. 4A, first jaw 114 opposes and is pivotally connected to second jaw 116. Device 100 includes a jaw manipulator 142 configured to manipulate opening and closing of tissue affecting device head 112 (i.e., relative pivotal positioning of jaws 114 and 116). Jaw manipulator 142 includes a longitudinal bar member 149 with a distal pore 143 (as shown in FIG. 4B). A distal end of jaw manipulator 142 may be threaded with a pin 152, via pore 143 through two tracks provided one about the other and defined by a first cam surface 114a and a second cam surface 116a, respectively. First cam surface 114a or/and second cam surface 116a may be continuous extensions or separate units joined with jaws 114 and 116, respectively. Jaw manipulator 142 is positioned and longitudinally extending within shaft body 120 and operatively connected to a control mechanism, such as control mechanism 18. By pulling or pushing jaw manipulator 142 the pin 152 is forced to travel through the tracks along first and second cam surfaces 114a and 116a, which in turn forces jaws 114 and 116 to open or close. Further description of the opening and closing mechanism via jaw manipulator 142 is provided herein below with reference to FIGS. 5A-5B.

Device 100 further includes a pivot shifting mechanism 154 configured to change relative (e.g., transaxial) positioning between first cam surface 114a and second cam surface 116a in a way that rearrange the jaws and their pivot, thereby facilitating device transformation from tissue dissecting configuration to tissue scissoring configuration, or vice versa, as previously described. In some embodiments, pivot shifting mechanism 154 is configured to selectively force jaws 114 and 116 to retract one from the other thereby creating a space accurately defined for allowing scissors blades 128 and 130 to slide in blade slots 138 within jaws 114 and 116, as previously described.

In an exemplary embodiment, pivot shifting mechanism 154 includes a device configuration selector 147 with opposing pivot shifting levers 147a and 147b which are selectively manipulatable with corresponding compressing manipulator projections 148a and 148b of a levers compressing manipulator 148, or with corresponding retracting manipulator projections 150a and 150b of a levers retracting manipulator 150. A combined action of device configuration selector 147 and levers compressing manipulator 148 is configured to enable required change for transforming from tissue dissecting configuration to tissue scissoring configuration. In an opposite fashion, a combined action of device configuration selector 147 and levers retracting manipulator 150 is configured to enable required change for transforming from tissue scissoring configuration to tissue dissecting configuration.

Shifting-lever 147a is pivotally connected with distal end thereof to jaw 114, optionally via a pin 145a, and pivotally connected with a proximal end thereof to shaft 120, optionally via a pin 145c. Shifting-lever 147b is pivotally connected with distal end thereof to jaw 116, optionally via a pin 145b, and pivotally connected with a proximal end thereof to shaft 120, optionally via a pin 145d. Each of shifting levers 147a and 147b has a proximally projecting extension that is shorter than lever length between the two pivot connections of the lever, such that by pressing together both lever extensions, any shift thereof with retract the jaws away from each other by way of greater magnitude. Optionally, alternatively or additionally, shifting levers 147a and 147b and jaws 114 and 116 may be uniformly fabricated from single piece of material.

By shifting distally the levers compressing manipulator 148 and shifting proximally the levers retracting manipulator 150, first manipulator projection 148a engages and rides over shifting lever 147a while second manipulator projection 148b engages and rides over shifting lever 147b, thereby pressing the two levers proximal extensions together, in a clamp-like manipulation, thereby moving jaws 114 and 116 apart onto the scissors configuration. Alternatively, by shifting proximally levers compressing manipulator 148 and shifting distally levers retracting manipulator 150, manipulator projection 150a engages and rides under shifting lever 147a while manipulator projection 150b engages and rides under shifting lever 147b, thereby retracting the two levers proximal extensions away from each other, thereby moving jaws 114 and 116 closer together onto the tissue dissecting configuration.

Levers retracting manipulator 150 is shown as a separate unit in FIG. 4C. Levers retracting manipulator 150 may include a half hollowed tubular member 155 distally connected with manipulator projections 150a and 150b. Levers compressing manipulator 148 is shown as a separate unit in FIG. 4D. Levers compressing manipulator 148 may include a half hollowed tubular member 151 distally connected with first and second manipulator projections 148a and 148b. Further description of the pivot shifting mechanism 154 and mode of operation is provided herein below with reference to FIGS. 5A-5B and FIGS. 6A-6D.

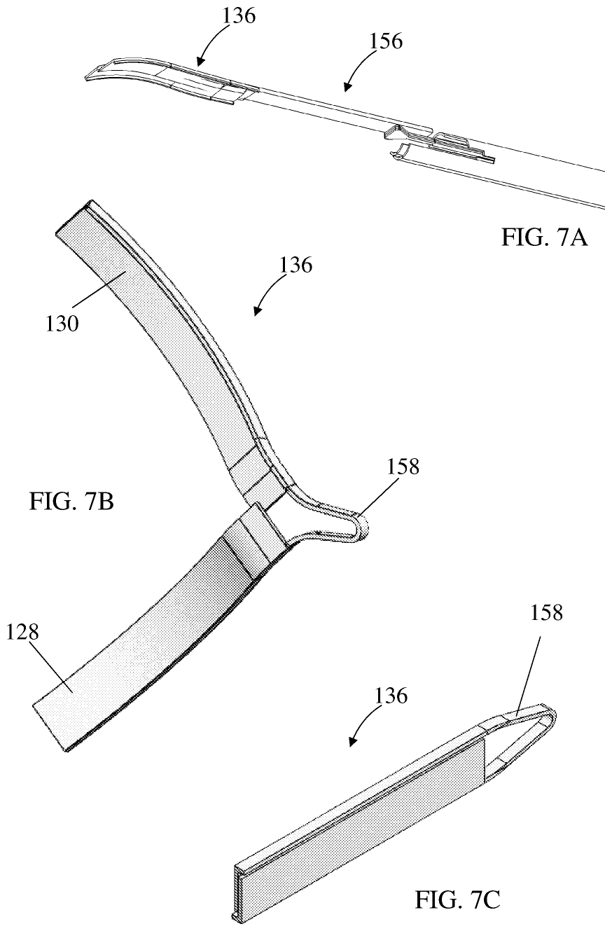

Device 100 may further include a scissors deploying mechanism 146 operably connected with a control mechanism, such as control mechanism 18 and configured for pushing and pulling each scissors blades 128 and 130 along a path and to thereby deploy and position the blades within jaws 114 and 116. Scissors deploying mechanism 146 extends within shaft 120 in juxtaposition to jaw manipulator 142, and includes a blades pusher member 156 connected with a distal end thereof to scissors blades 128 and 130. Blades pusher member 156 is shown as a separate unit in FIG. 4E. Reference is now made to FIGS. 7A-7C demonstrating blades pusher member 156 in the form of a longitudinal rod. Blades pusher member 156 is connected with a distal end thereof to each scissors blades 128 and 130, and configured for pushing and pulling each scissors blades 128 and 130 along a path within shaft 120 of device 100. Scissors blades 128 and 130 may be proximally connected with a flexible link 158. According to some embodiments, scissors blades 128 and 130 are formed as a shear-like insert.

As shown in FIGS. 5A-5B, tissue affecting device head 112 opening is effected when jaw manipulator 142 is pushed forward towards the jaws in longitudinal distal direction. Closing of tissue affecting device head 112 is effected when jaw manipulator 142 is pulled backward in longitudinal proximal direction. Push (in a distal direction) and pull (in a proximal direction) of jaw manipulator 142 is optionally achieved and controlled manually by a surgeon when manipulating control mechanism 18, optionally via a lever arm (such as lever arm 24, shown in FIG. 1A, for example), or any other switching or actuating means.

According to some embodiments, distal and proximal movements of jaw manipulator 142 actuates pivot shifting mechanism 154 to shift pin 152 relative to at least one of cam surfaces 114a and 116a. According to some embodiments, movement in a distal direction of jaw manipulator 142 effects distal shift of pin 152 relative to cum surfaces 114a and 116a, converting the device to the opened state (shown in FIG. 5B). According to some embodiments, lateral movement in a proximal direction of jaw manipulator 142 effects proximal shift of pin 152 relative to cum surfaces 114a and 116a, converting the device to the closed dissector state (as shown in FIG. 5A).

According to some embodiments, the closed state allows contact or close proximity between dissector jaws 114 and 116. According to some embodiments, in the opened state, jaws 114 and 116 are spaced apart with force required to dissect tissues.

Reference is now made to FIGS. 6A-6D demonstrating transformation of device 100 from the tissue dissecting configuration to the tissue scissoring configuration. Transformation to tissue scissoring configuration is controllably effected by a surgeon via control mechanism, optionally by operating a dedicated actuator, such as actuator 26 (illustrated in FIG. 1A), for example.

Transformation to tissue scissoring configuration includes spacing between jaws 114 and 116 to allow place to scissors bladed 128 and 130 to slide and lock within jaws 114 and 116. Jaws 114 and 116 may be spaced apart in parallel such that jaws 114 and 116 move in opposite directions, wherein jaw 114 is moved in an upward direction and jaw 116 is moved in a downward direction.

In some embodiments, by changing between tissue dissecting configuration and tissue scissoring configuration, as previously described, pivot shifting mechanism 154 shifts pin 152 to a respective position between cam surfaces 114a and 116a, from a first travel range (or position), allowing the jaws 114 and 116 to close up to a contact or close proximity therebetween (as shown in FIGS. 6A-6B), to a second travel range (or position), in which the jaws preserve a minimally allowed space therebetween (as shown in FIGS. 6C-6D). Afterwards, blades pusher member 156 can be actuated to push each scissors blades 128 and 130 along a path and via slot 138 (shown in FIGS. 3A-3B) affording locking, and optionally curving, within jaws 114 and 116.

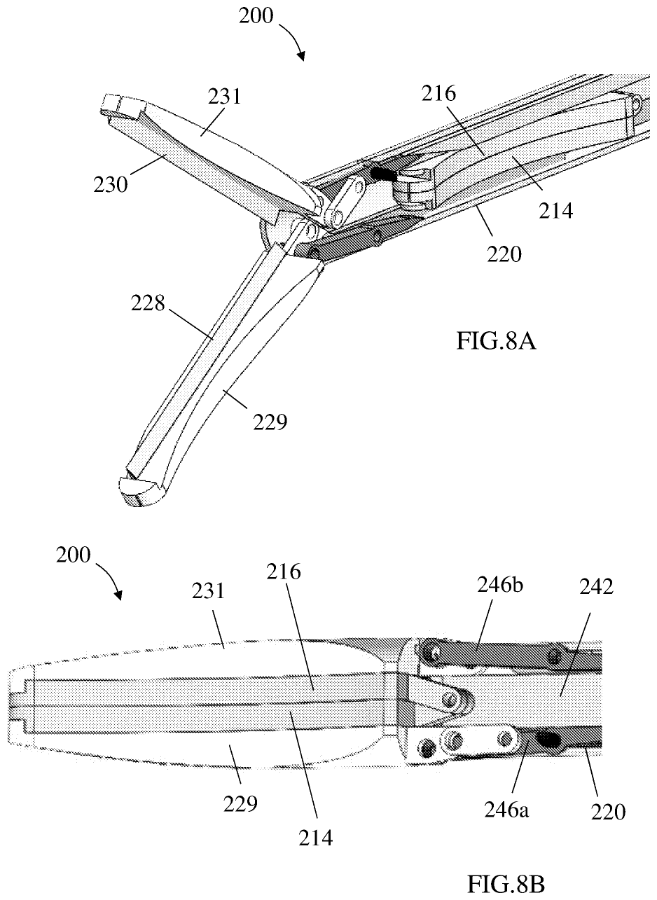

FIG. 8A-8B show another exemplary device 200 of the invention which may be considered an inverted form of devices 10 or 100. Device 200 includes a first scissors blade 228 and a second scissors blade 230 that are operable to effect a tissue cutting. First scissors blade 228 is (permanently) affixed to a first jaw 229, and second scissors blade 230 is (permanently) affixed to a second jaw 231, and jaws 229 and 231 are pivotally connected and actuatable using actuating means such as those described with respect to devices 10 and 100. Jaws 229 and 231, with scissors blades 228 and 230, are shaped and configured to accommodate and locked therewith dissecting inserts 214 and 216, respectively. Similarly to devices 10 and 100, device 200 is transformable between a tissue scissoring configuration and a tissue dissecting configuration and vice versa, although in opposite manner.

FIG. 8A shows a proximal portion of device 200 of the invention in the scissors configuration, wherein dissecting inserts 214 and 216 are retracted and confined within a shaft 220. FIG. 8B shows a proximal portion of device 200 of the invention, in the tissue dissecting configuration wherein dissecting inserts 214 and 216 are deployed over blades 228 and 230, respectively, and fixedly connected to jaws 229 and 231, respectively. According to some embodiments, opening and closing of jaws 229 and 231 (either as scissors head or as dissector head) is effected similarly to the opening and closing mechanism of devices 10 and 100.

In accordance with this embodiment, jaw manipulator 242 movement in a distal direction effects scissors head 236 or dissector head 212 opening while jaw manipulator 242 movement in a proximal direction effects scissors head 236 and dissector head 212 closing. According to further embodiments, transformation from the tissue scissoring configuration to the tissue dissecting configuration and vice versa are effected similarly to the transformation mechanisms of devices 10 or 100. In accordance with this embodiment, a pivot shifting mechanism (similar to the pivot shifting mechanism 154 of device 100) that facilitates spacing between blades 228 and 230 is controllably and selectively actuated, optionally via opposing pivot shifting levers 247a and 247b. Also optionally, a scissors deployment mechanism (similar to the scissors deployment mechanism 146 of device 100) is controllably and selectively actuated to mediate sliding and locking of dissector jaws 214 and 216 within blades 228 and 230, respectively.

The present invention further provides a system that includes a surgical device as described herein and a tissue grasping apparatus. The tissue grasping apparatus may be provided as a separate unit that can be integrally combined with the surgical device of the invention, thereby forming one instrument that can be controlled via a singular control mechanism. Alternatively, the tissue grasping apparatus can be provided as a separate unit that is controlled via a separate control mechanism. Further alternatively, the tissue grasping apparatus is integrally manufactured with the surgical device of the invention.

Some embodiments of the invention refer to a system, wherein the tissue grasping apparatus and the surgical device may be provided as an assembly that can be controlled by a singular and same control mechanism, and that can be disassembled such that each of the tissue grasping apparatus and the surgical device can be also operated autonomously via a control unit provided for each of those instruments. In view of the above the control mechanism of each the tissue grasping apparatus and the surgical device can be operated autonomously or/and when combined into a singular control mechanism or unit.

According to an aspect of some embodiments of the present invention, there is provided a system for performing surgical interventions in a body of a subject. In exemplary embodiments, the system includes: a surgical device including a longitudinal shaft upon which is mounted a tissue affecting device head; a tissue grasping apparatus including a hollow grasping head having pivotally connected first and second grasping jaws configured for grasping tissue; a tissue grasping manipulator configured for forcibly manipulating the first and second grasping jaws from an opened state to a closed state, such that the hollow grasping head in the closed state forms a grasping head lumen sized and shaped to allow the tissue affecting device head to pass therethrough and therealong; and a shifting mechanism configured for shifting the surgical device to an operative position within, or distal to, the hollow grasping head.

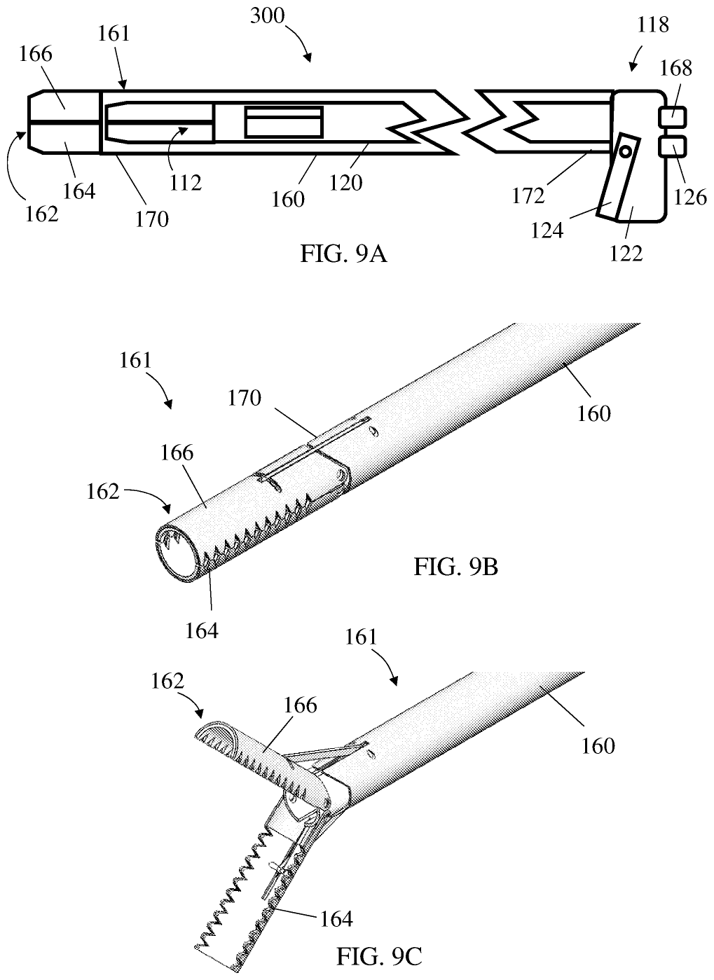

Referring to FIGS. 9A-9C demonstrating and exemplary system 300 of the invention that includes end effector capabilities with replaceable means including a grasper, a dissector and scissors, each can be selectively assembled or/and fixed as a single end effector, and be controlled using same control mechanism actuatable by a user from outside subject's body. In some such embodiments, the system embeds a surgical device, such as surgical device 100, encapsulated in a tissue grasping apparatus 161, both connected to each other, controllable and actuatable using a singular control mechanism 118.

System 300 is presented herein below with reference to device 100 of the invention but can be operated with any one of the surgical devices of the invention.

Tissue grasping apparatus 161 is configured for affecting a tissue by grasping or/and pressing a tissue, optionally prior to or/and following dissecting or/and cutting the tissue (e.g., using dissector/scissors provided embedded in the system). Tissue grasping apparatus 161 may be configured for affecting hard materials and tissues such as bones, or/and for holding instrumentation in the body of the treated subject. Tissue grasping apparatus 161 may be designed to serve as a conduit (in a form of port or cannula) through which the device 100 or other instruments may be inserted to or removed from within the subject's body. Tissue grasping apparatus 161 may be integrally connected and manufactured as part of device 100. Alternatively, tissue grasping apparatus 161 may be provided as a separate device with special integrating means with device 100. In accordance with those embodiments, device 161 may be provided either separately or as part of a kit further including device 100.

Tissue grasping apparatus 161 includes an outer tube 160 extending from an outer tube distal end 170 and an outer tube proximal end 172. Outer tube 160 is sized and shaped to accommodate dissector shaft 120 (and dissector head 112 attached to shaft 120). Outer tube 160 is further sized for allowing dissector shaft 120 and dissector head 112 sliding therethrough and along a longitudinal axis thereof. Outer tube 160 includes a hollow grasping head 162, having opposing pivotally connected grasping jaws 164 and 166. According to some embodiments, outer tube 160 connects between control mechanism 118 and grasping head 162. Optionally, tissue grasping manipulator 168 disposed within control mechanism 118 is provided for controlling grasping head 162 closing (as shown in FIG. 9B) or/and opening (as shown in FIG. 9C). According to some embodiments, at least one of grasping jaws 164 and 166 is formed as a half-tube. According to some embodiments, at least one of grasping jaws 164 and 166 includes a toothed edge allowing tissue grasping.

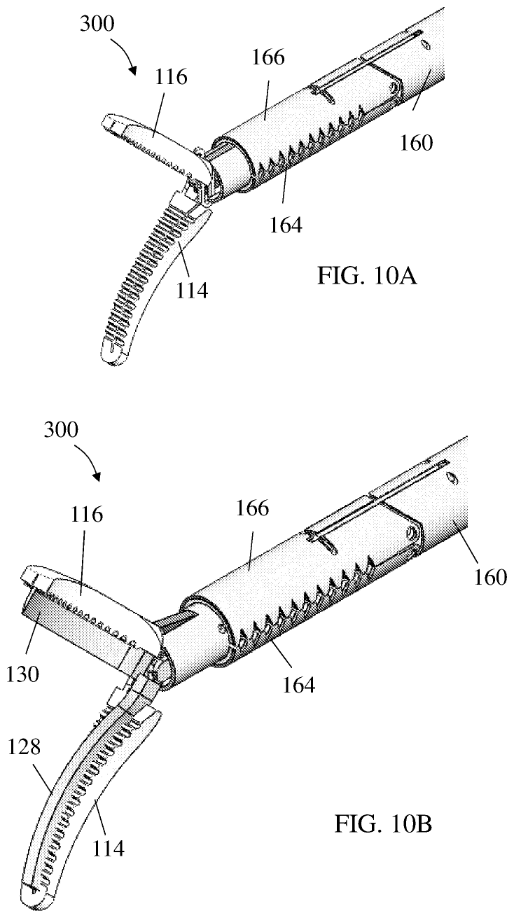

Referring to FIGS. 10A-10B, retraction of outer tube 160 and operation of device 100 in the tissue dissecting configuration is shown in FIG. 10A. Retraction of outer tube 160 and operation of tissue scissoring configuration of device 100 is shown in FIG. 10B. In the tissue scissoring configuration, scissors blades 128 and 130 are deployed and disposed within jaws 114 and 116, respectively.

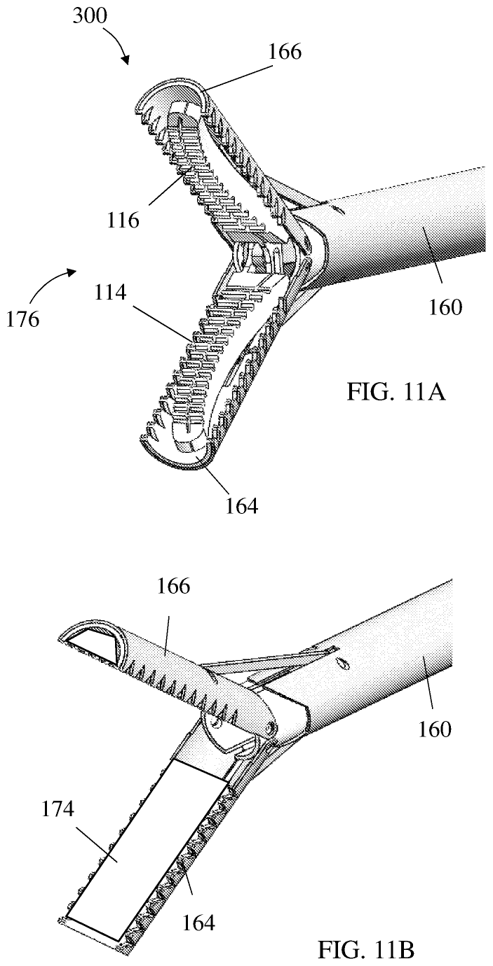

FIG. 11A shows a distal portion of system 300 of the invention having a tissue grasping apparatus 161 including an outer tube 160 and grasping head 162 pivotally connected and extending from a distal end of outer tube 160. Grasping head 162 encloses a lumen sized for accommodating dissector head 112 therein, thereby allowing combined operation of grasping head 162 and dissector head 112 as an integral grasper head 176. According to some embodiments, opening and closing integral grasper head 176 is effected using same mechanism used for opening and closing dissector head 112. According to alternative embodiments, opening and closing grasping integral grasper head 176 is effected via tissue grasping manipulator 168 disposed within control mechanism 118.

FIG. 11B shows an exemplary embodiment of the invention, wherein at least one of grasping jaws 164 and 166 is filled with a viscoelastic or partially filled malleable component 174. Component 174 is configured to at least partially conform to a corresponding dissector jaw 114 or 116 submerging therein, thereby facilitating an enlarged contact area for grasping, or/and even distribution of compression stresses, when applying integral grasper head 176 for grasping a soft tissue.

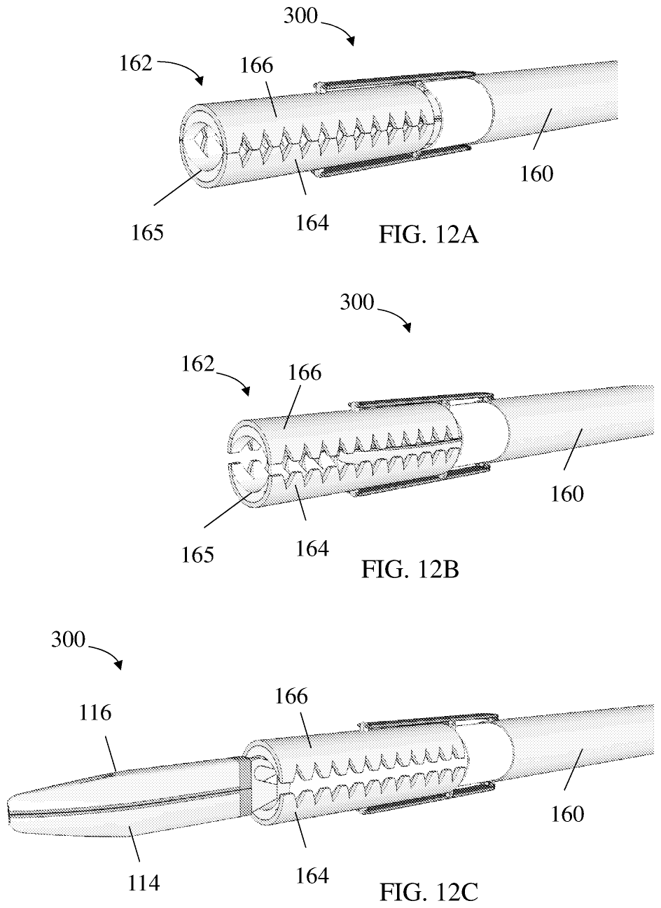

FIGS. 12A-12C show a distal portion of system 300 of the invention, wherein at least one of grasping jaws 164 and 166 includes a sloped surface 165 shaped and dimensioned such that dissector head 112, when engaging and pushing against the sloped surface 165, forces grasping jaws 164 and 166 to retreat radially sufficiently for allowing dissector head 112 advancing across a distal end of the grasping head 162. FIG. 12A shows grasping head 162 in a closed state, wherein dissector head 112 and shaft 120 are retracted within or separated from outer tube 160. FIG. 12B shows grasping head 162, wherein jaws 164 and 166 are partially opened and dissector head 112 and shaft 120 advance to emerge from grasping head 162. FIG. 12C shows grasping head 162 when retracted radially and following dissector head 112 insertion through or/and advancing across outer tube 160.

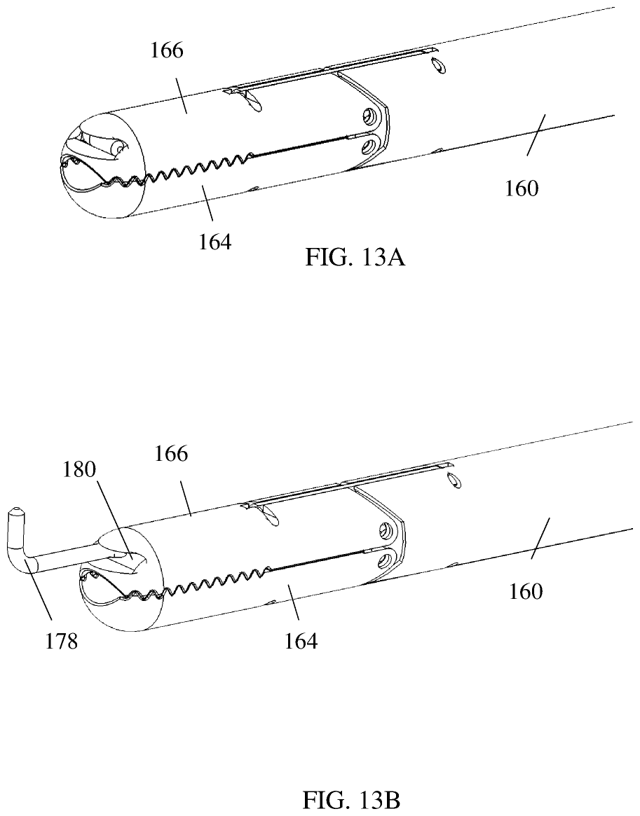

FIGS. 13A-13B show a distal portion of tissue grasping apparatus 161 of the invention, wherein the device further includes a cautery electrode 178. Cautery electrode 178 may be provided and configured to effect tissue manipulation, selected from, but not limited to: tissue cutting, tissue coagulation, or tissue desiccation. Cautery electrode 178 may be connected with any suitable cautery means to any suitable position within, or portion of, tissue grasping apparatus 161. Cautery electrode 178 may be located in a position within any one or both jaws 164 and 166. Alternatively, additionally or optionally, cautery electrode 178 may be located in a position within outer tube 160. In an exemplary embodiment, cautery electrode 178 may be deployed within tissue grasping apparatus 161 (as shown in FIG. 13A) and extendable from a recess 180 in grasping head 162 (shown in FIG. 13B). In a further exemplary embodiment, cautery electrode 178 is forced to revolve from a horizontal position to a vertical position when extending from recess 180 to a fully deployed position (shown in FIG. 13B). Further optionally, cautery electrode 178 is in a form of a hook.

Some embodiments of the invention refer to applications (methods) for using the herein disclosed surgical devices and systems.

According to an aspect of some embodiments of the invention, there is provided a method for performing surgical interventions in a body of a subject. In exemplary embodiments, the method includes: introducing, through an incision in the body, a surgical device having a tissue affecting device head extending from a distal end of a longitudinal shaft and having pivotally connected first and second dissector jaws, and further having a first scissors blade and a second scissors blade each sized and configured for sliding, and locking within a corresponding one of the first and second dissector jaws, wherein the tissue affecting device head is reversibly convertible between a tissue dissecting configuration and a tissue scissoring configuration, and between an opened state and a closed state via a control mechanism extending from proximal end of the longitudinal shaft.

In exemplary embodiments, the method further includes dissecting tissue using the surgical device when the tissue affecting device head is in the tissue dissecting configuration, and, by opening the tissue affecting device head and closing the tissue affecting device head around the tissue.

In exemplary embodiments, the method further includes converting the tissue affecting device head into the tissue scissoring configuration by the sliding and locking the first and second scissors blades within the first and second dissector jaws.

In exemplary embodiments, the method further includes cutting the tissue by opening the tissue affecting device head and closing the tissue affecting device head around the tissue.

In exemplary embodiments, the method further includes introducing a tissue grasping apparatus via the incision, wherein the tissue grasping apparatus includes an outer tube sized to accommodate and facilitate sliding therethrough the longitudinal shaft and the tissue affecting device head, wherein the outer tube is connected via a distal end thereof to a hollow grasping head having pivotally connected first and second grasping jaws, and the grasping head is forcibly transformable between an opened state and a closed state.

In exemplary embodiments, and in a non-limiting manner, device 100 and system 300 are suitable for being used in various interventional procedures in a body.

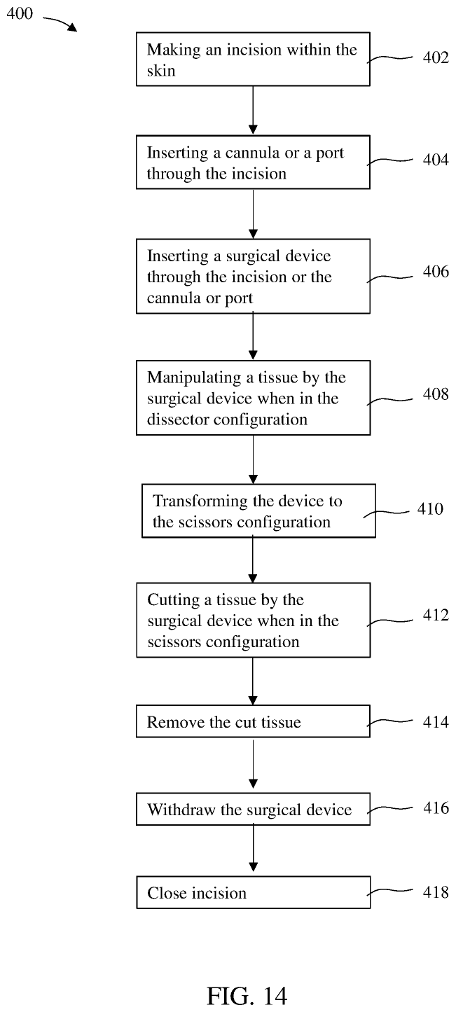

Reference is now made to FIG. 14 depicting an exemplary method 400 of performing an interventional procedure within the body of a subject. A surgical device is provided that may be similar or identical to any of the abovementioned devices. For illustrative purposes only, the method of the invention is described with reference to device 100 and system 300 described herein, but is applicable with any of the above mentioned devices or/and systems. Method 400 includes one or more steps (procedures) as shown in FIG. 14.

At step (procedure) 402 a relatively small incision is firstly made in the skin of the subject. At step (procedure) 404, a cannula, a port or any of the alike is inserted through the incision to allow convenient insertion of the device 100 or/and where applicable insertion of additional instruments. In some embodiments, tissue grasping apparatus 161 is inserted through the incision to facilitate therethrough insertion of the device 100. In some embodiments, the device 100 is inserted directly within the incision without any cannula or port.

At step (procedure) 406, the surgical device 100 of the invention is inserted through the incision and positioned proximate an area inside the body upon which a surgical procedure is required. In typical cases, device 100 is inserted within the body when in the tissue dissecting configuration and in a closed state (as shown, for example in FIG. 4A). In this configuration, the scissors head is maintained retracted within the shaft 120 (as shown, for example in FIG. 4A). Device 100 manipulation for dissecting a tissue is performed via a control mechanism, similar to control mechanism 18 shown in FIGS. 1A-1D which controls opening and closing of tissue affecting device head 112.

At step (procedure) 408, the surgeon may operate the device to tear, cut or dissect a tissue by opening and closing tissue affecting device head 112 in its dissecting configuration. In an exemplary embodiment, tissue can be dissected following opening the device head, for example by manipulating lever arm 124 upward (away from the handle) and placing the tissue between jaws 114, and 116. The device head 112 may then be closed around the tissue by manipulating lever arm 124 downwards.

At step (procedure) 410 the device 100 is transformed to the scissoring configuration. When in the scissoring configuration, the device head can be operated to cut soft tissue upon manipulating its control mechanism (either 18 or depending on the system used), optionally via actuator such as 26 or 126 (e.g., by pressing actuator 126). The scissors configuration is set upon deploying scissors blades 128 and 130, within jaws 114 and 116, respectively. Scissors blades 128 and 130 deployment may be mediated utilizing pivot shifting mechanism 154 or/and scissors deployment mechanism 146, optionally by remotely pressing actuator 126 of control mechanism 118. Pivot shifting mechanism 154 facilitates spacing between dissector jaws 114 and 116 by shifting a position between cam surfaces 116a and 114a, from a first travel range (FIGS. 6A-6B) to a second travel range (FIGS. 6C and 6D). Scissors deployment mechanism 146 facilitates sliding, optionally curving, and locking of scissors blades 128 and 130 within jaws 114 and 116, thereby assembling a scissors blades 136 that may effectively cut a tissue. According to some embodiments, the steps of: spacing between dissector jaws 114 and 116, sliding scissors blades 128 and 130 along a path within jaws 114 and 116, and locking scissors blades 128 and 130 within jaws 114 and 116, may occur sequentially or separately by manipulating one or more components (e.g., pressing actuator 126) within control mechanism 18.

At step (procedure) 412 tissue can be cut. Tissue can be cut in the scissoring configuration optionally, by utilizing same mechanism used for opening and closing device head 112. That is to say, by manipulating jaw manipulator 142 to move in a distal direction, thereby effecting opening and in a proximal direction, thereby effecting closing of jaws 114 and 116. When tissue cutting is accomplished, the scissors head 136 can once again be retracted and positioned within shaft 120.

At step (procedure) 414, the cut tissue may then be removed and the device is transformed back to the tissue dissecting configuration or/and withdrawn from the body. Where applicable, further instruments may be inserted (optionally through the cannula, port or tissue grasping apparatus 161) for performing further tissue manipulations, (e.g., suturing and or coagulating a tissue).

At steps (procedures) 416 and 418, the device is removed and the incision is closed.

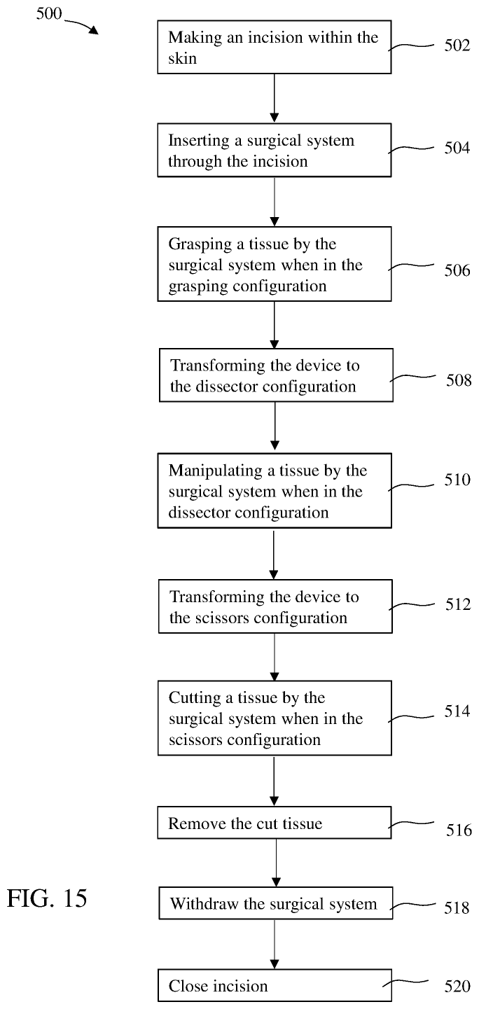

The present invention further affords a method 500 as depicted herein below and in FIG. 15, wherein the tissue grasping apparatus 161 is manipulated and actuated to grasp a tissue. As detailed above, tissue grasping apparatus 161 may include an outer longitudinal hollowed tube 160 connected at the distal end 170 to a grasping head 162. Tissue grasping apparatus 161 is sized and shaped to accommodate device 100 when in the closed state (as shown, for example in FIG. 4A). The method 500 includes one or more steps (procedures) as depicted in FIG. 15.

At step (procedure) 502, a small incision is made in the skin of the subject.

At step (procedure) 504, system 300 is inserted within the incision. The system is inserted in the grasping configuration, wherein the grasping head 162 is in a closed state.

At step (procedure) 506, tissue grasping apparatus 161 is operated for grasping a tissue, either as an autonomous unit or via integral grasper head 176 that combines grasping head 162 and dissector head 112. Tissue grasping is effected by opening and closing grasping head 162 or grasper head 176 around a tissue.

At step (procedure) 510, there is manipulating tissue using system 300 when in the tissue dissecting configuration. To this end, the system is transformed, as depicted at step (procedure) 508, to the dissecting configuration. This is accomplished by retracting outer tube 160 and advancing device 100 within outer tube 160. For tissue cutting, the system is transformed, as indicated by step (procedure) 512, to the scissoring configuration.

At step (procedure) 514, there is cutting a tissue by system 300 when in the scissoring configuration. The cut tissue may be removed as depicted in step 516 from the body.

It is to be noted that the tissue grasping apparatus 161 may perform tissue grasping or other tissue manipulations prior to or following any one of steps (procedures) 506 to 516. For example, tissue grasping apparatus 161 may perform at least one of tissue cutting, tissue coagulation, or tissue desiccation, via operating a cautery electrode 178, that may be disposed within a position on tissue grasping apparatus 161. Cautery electrode may be remotely operated when manipulating an element within control mechanism 118 or within tissue grasping apparatus 161. Further optionally and where applicable, additional instruments may be inserted (optionally through the tissue grasping apparatus 161) for performing further tissue manipulations.

At step (procedure) 518, there is closing the incision by suturing the opening, for example, using techniques known in the art.

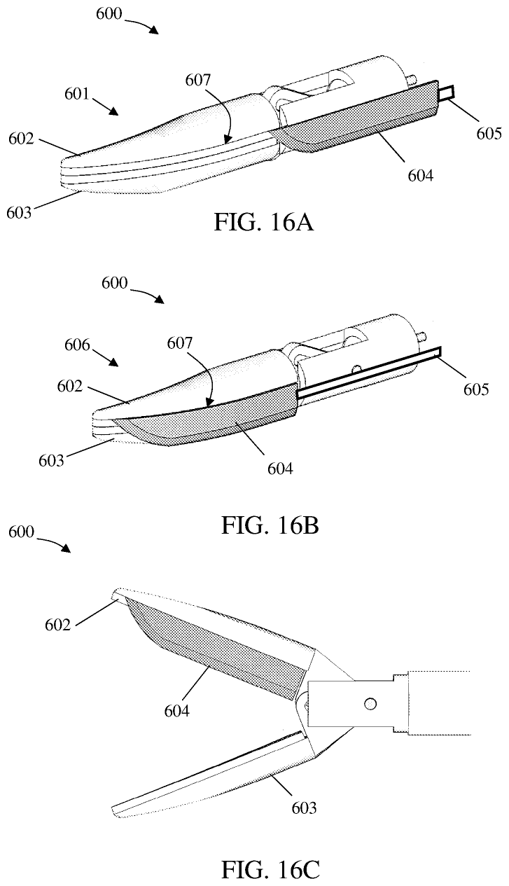

FIGS. 16A-16C are respective views of an exemplary device 600 of the invention, wherein a tissue affecting device head transforms from a dissector head 601 configuration to a cutting head 606 configuration, by integrating with a side blade 604. As shown in FIG. 16A, device 600 is provided or deliverable into the body with side blade 604 retracted proximally to dissector head, such that dissector jaws 602 and 603 of dissector head 601 can be pivotally shifted one relative to the other for grasping or dissecting a tissue, as previously described for devices 10 and 100, for example. Once cutting functionality is required, side blade 604 can be pushed using a blade pusher 605 until it is slides and locked in place in a side slot 607 as shown in FIG. 16B. Side blade 604 can be used to cut or resect tissue while progressing forward (distally). Optionally, alternatively or additionally (and as shown in FIG. 16C), once locked in final position in slot 607, can be be used for cutting or scissoring with pivotal motion of the jaws 602 and 603.

According to an aspect of some embodiments of the present invention, there is provided a surgical device including: a longitudinal shaft enclosing a lumen having a cross section with a curved boundary; and a plurality of surgical instruments provided in the lumen, wherein at least two of the surgical instruments are packed according to a nested or/and concentric arrangement relative to the lumen curved boundary. In exemplary embodiments, each of the plurality of surgical instruments is configured for being selectively, slidably protrudable from the remainder of the surgical instruments, into a deployable form, thereby facilitating use of the deployable form for performing a single surgical procedure (maneuver), or for performing a set of two or more surgical procedures (maneuvers). According to some embodiments of the invention, the deployable form is configured for performing a plurality of different types of single surgical procedures (maneuvers), or a plurality of different types of sets of two or more surgical procedures (maneuvers).

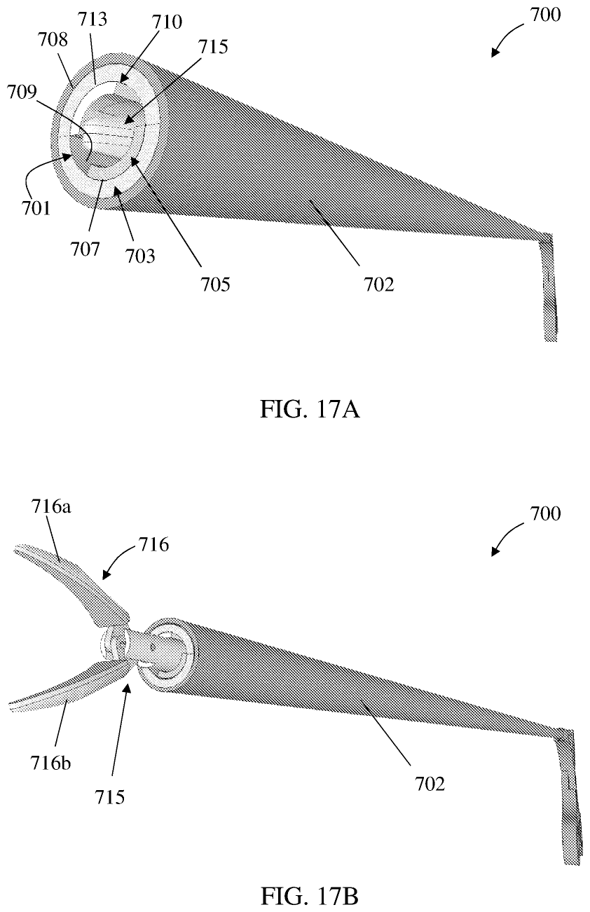

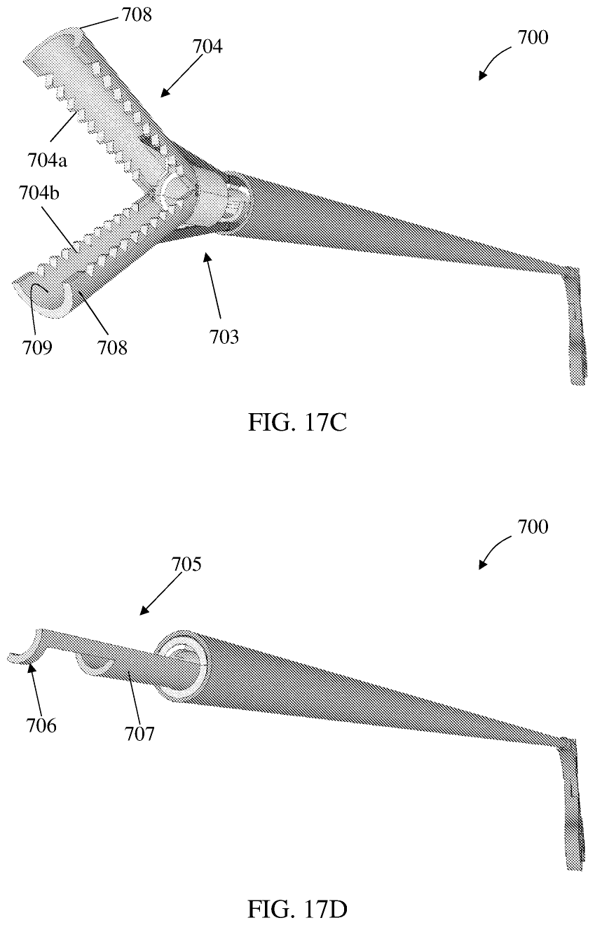

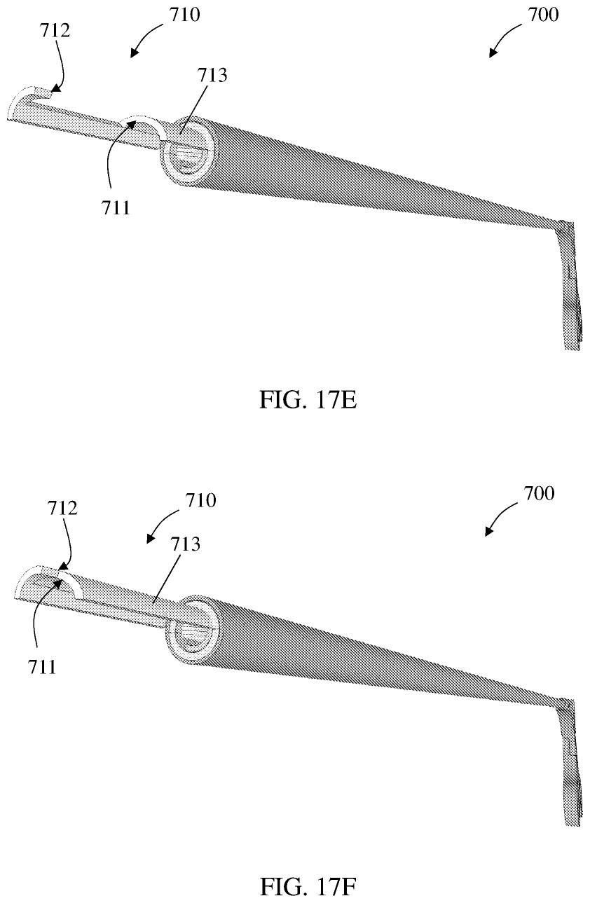

Reference is now made to FIGS. 17A-17F showing respective views of an exemplary device 700 which includes a plurality of surgical instruments packed in a lumen 701 of a longitudinal shaft 702 of the device 700. Shaft lumen 701 has substantially curved (e.g., circular) boundary hence in order to pack as many possible surgical instruments thereinside, advantage may be found in having at least some surgical instruments packed in a nested or/and concentric arrangement. In some such embodiments, at least some surgical instruments are provided as concentric ‘layers’ with an ‘outer’ instrument inner periphery lay in direct contact with outer periphery of an ‘inner’ instrument. The term “nested” in this embodiment refers to at least two distinct members or/and instruments, configured such that one fits inside the other, both being curved substantially similar or same, in at least two facing portions thereof, thereby allowing close proximity or even direct of surfaces thereof. The term “concentric” in this embodiment refers to at least two distinct members or/and instruments having common center in at least two facing portions thereof. In some such embodiments, each surgical instrument is selectively slidably protrudable to the rest into a deployable form, then it can be applied to perform a particular surgical procedure (maneuver), or set of surgical procedures (maneuvers), optionally being substantially different or/and distinctable to other surgical procedures (maneuvers) or sets of surgical procedures (maneuvers) applicable by the other surgical instruments.

In some embodiments, the elongated shaft 702 is sized for entry at laparoscopic/key-hole surgery openings into a subject body (e.g., via a pre-made incision or pre-installed sheath), and optionally it has a maximal outer diameter of about 15 mm or less, optionally about 12 mm or less, optionally about 8 mm or less, optionally about 5 mm or less, or optionally about 3 mm or less. In some embodiments, inner diameter of of shaft 702 (i.e., shaft lumen 701 diameter) is 0.1 to 1 mm smaller than its outer diameter, and optionally between 2 mm and 6 mm, optionally between 3 mm and 5 mm.

FIG. 17A shows device 700 with a number of surgical instruments, all extending entirely within shaft lumen 702. Surgical instruments may include at least two, optionally at least three, of a grasper, a scissors, a hook, a cutter, and a dissector. Exemplary embodiment shown in FIGS. 17 includes a grasper 703, a hook 705, a cutter 710 and a dissector 715, all configured to packing within shaft lumen 701 and selectively deployed, each, to a protruding position therefrom.

Grasper 703, also shown in FIG. 17C in a protruding (deployed) position, includes a tubular grasper head 704 having two opposing half-tube shaped jaws 704a and 704b. Tubular grasper head 704 has a convex (outer) periphery 708 fitting (nesting) in close proximity or even direct contact with inner periphery of shaft lumen 702.

Hook 705, also shown in FIG. 17D in a protruding (deployed) position, has a hook terminal 706 for manipulating or/and affecting a tissue, which may or may not include an electrode or otherwise for heating or coagulating soft tissues. Hook 705 is sized and shaped (curved) with a convex (outer) surface 707 configured to nest within a concave (inner) periphery 709 of grasper head 704. Optionally and as shown, the circumference (length) of hook 705 along its curvature is optionally equal to or less than circumference of grasper head 704, optionally equal to or less than circumference of any of its jaws 704a or 704b.

Cutter 710, also shown in FIGS. 17E and 17F in a protruding (deployed) position, is optionally structured and configured to function similarly to a guillotine, for cutting, scissoring or dissecting soft tissues. Cutter 710 includes a slidable blade 711 and a stop 712, such that a soft tissue, if entrapped between blade 711 and stop 712 when blade 711 is situated away (withdrawn) from stop 712 (FIG. 17E), can be cut upon blade 711 sliding across the soft tissue towards stop 712, until possible direct contact (FIG. 17F). Scissoring may be accomplished in case that stop 712 also includes a sharp edge which is slightly spaced away from blade 711 when it is in contact with stop 712. Cutter 710 is sized and shaped (curved) with a convex (outer) surface 713 configured to nest within a concave (inner) periphery 709 of grasper head 704. Optionally and as shown, the circumference (length) of cutter 710 along its curvature is optionally equal to or less than circumference of grasper head 704, optionally equal to or less than circumference of any of its jaws 704a or 704b.

Dissector 715, also shown in FIG. 17B in a protruding (deployed) position, includes a dissector head 716 with two opposing dissector jaws 716a and 716b, being each relatively slender and may be moderately curved as known in the art with respect to surgical soft tissue dissectors. Having its jaws closed (as shown in FIG. 17A), dissector head 716 is sized and shaped such that dissector 715 can be fully withdrawn inside remaining space formed by other surgical instruments provided in lumen 701 of a longitudinal shaft 702 of the device 700. As shown in this example, dissector 715 is located in the center of lumen 701 encircled with rest of surgical instruments, although any other arrangement with similar packing (fitting/nesting) arrangement may be applied instead, under provisions of this invention.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of.’Each of the phrases ‘consisting of’ and ‘consists of’, as used herein, means ‘including and limited to.’

The term ‘method’, as used herein, refers to a single step, procedure, manner, means, or/and technique, or a sequence, set, or group of two or more steps, procedures, manners, means, or/and techniques, for accomplishing or achieving a given task or action. Any such herein disclosed method, in a non-limiting manner, may include one or more steps, procedures, manners, means, or/and techniques, that are known or readily developed from one or more steps, procedures, manners, means, or/and techniques, previously taught about by practitioners in the relevant field(s) and art(s) of the herein disclosed invention. In any such herein disclosed method, in a non-limiting manner, the stated or presented sequential order of one or more steps, procedures, manners, means, or/and techniques, is not limited to that specifically stated or presented sequential order, for accomplishing or achieving a given task or action, unless otherwise specifically defined or stated herein, or, unless the context clearly dictates otherwise. Accordingly, in any such herein disclosed method, in a non-limiting manner, there may exist one or more alternative sequential orders of the same steps, procedures, manners, means, or/and techniques, for accomplishing or achieving a same given task or action, while maintaining same or similar meaning and scope of the herein disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’

The term ‘about’, as used herein, refers to ±10% of the stated numerical value.

The phrase ‘operatively connected’, as used herein, equivalently refers to the corresponding synonymous phrases ‘operatively joined’, and ‘operatively attached’, where the operative connection, operative joint, or operative attachment, is according to a physical, or/and electrical, or/and electronic, or/and mechanical, or/and electro-mechanical, manner or nature, involving various types and kinds of hardware or/and software equipment and components.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, are encompassed by the broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A surgical device for performing surgical interventions in a body of a subject, the device comprising:
a tissue affecting device head extending from distal end of a longitudinal shaft and including a first dissector jaw connected to a second dissector jaw with a pivot, each of said dissector jaws includes a slot extending therealong, a first scissors blade sized and configured for sliding, and locking into a first said slot in said first dissector jaw, and a second scissors blade sized and configured for sliding, and locking into a second said slot in said second dissector jaw;
a jaws manipulator, configured for manipulating said first and second dissector jaws; and
a control mechanism operatively connected, with said jaws manipulator, to said tissue affecting device head, and configured to selectively shift said dissector jaws from a closed state into an opened state;
wherein said tissue affecting device head is reversibly convertible between a tissue dissecting configuration, whereby said tissue affecting device head is structurally and functionally configured for dissecting soft tissue, and a tissue scissoring configuration, whereby said tissue affecting device head is structurally and functionally configured for scissoring soft tissue;
wherein said pivot, said control mechanism, and said jaws manipulator are operatively connected to said tissue affecting device head for facilitating operation of, and said reversible convertibility between, said tissue dissecting configuration and said tissue scissoring configuration, whereby actuation force is transferrable to said dissector jaws in said tissue scissoring configuration, and whereby actuation force is also transferrable to said scissors blades via said dissector jaws.

2. A surgical device according to claim 1, wherein, in said tissue dissecting configuration said scissors blades are disposed within said longitudinal shaft, and in said tissue scissoring configuration said scissors blades are locked within said first and second dissector jaws.

3. A surgical device according to claim 1, further comprising a pivot shifting mechanism configured to selectively shift said pivot relative to a cam surface, from a first pivot travel range that allows contact between said first and second dissector jaws to a second pivot travel range, thereby facilitating spacing between said first and second dissector jaws.

4. A surgical device according to claim 3, wherein said pivot shifting mechanism includes a device configuration selector configured for said spacing between said first and second dissector jaws, so as to provide space for said scissors blades to slide therein, and for driving said scissors blades to slide and lock in said first and second slots.

5. A surgical device according to claim 1, further comprising a scissors deploying mechanism operatively connected with said control mechanism and including a blades pusher member connected with a distal end thereof to each one of said scissors blades, and configured for pushing and pulling each one of said scissors blades along a path.

6. A surgical device according to claim 1, wherein said first dissector jaw is curved with said first slot extending and curved therealong and said second dissector jaw is curved with said second slot extending and curved therealong.

7. A surgical device according to claim 1, wherein said control mechanism includes a handle disposed at proximal end of said longitudinal shaft, and is configured for selectively actuating said jaws manipulator.

8. (canceled)

9. A device according to claim 1, wherein said control mechanism is positioned at proximal end of said longitudinal shaft, and includes a robotic arm configured for selectively actuating said jaws manipulator.

10. A surgical device according to claim 1, wherein said scissors blades are connected to each other with a flexible link.

11. A surgical device according to claim 1, wherein said scissors blades are formed as a shear-like insert.

12. A surgical device according to claim 1, connectable to or integratable into a system for performing surgical interventions in a body of a subject, the system further comprising:
a tissue grasping apparatus including a hollow grasping head having pivotally connected first and second grasping jaws configured for grasping tissue;
a tissue grasping manipulator configured for forcibly manipulating said first and second grasping jaws from an opened state to a closed state, such that said hollow grasping head in said closed state forms a grasping head lumen sized and shaped to allow said tissue affecting device head to pass therethrough and therealong; and
a shifting mechanism configured for shifting said surgical device to an operative position within, or distal to, said hollow grasping head.

13. A system for performing surgical interventions in a body of a subject, the system comprising:
a surgical device including a longitudinal shaft upon which is mounted a tissue affecting device head;
a tissue grasping apparatus including a hollow grasping head having pivotally connected first and second grasping jaws configured for grasping tissue;
a tissue grasping manipulator configured for forcibly manipulating said first and second grasping jaws from an opened state to a closed state, such that said hollow grasping head in said closed state forms a grasping head lumen sized and shaped to allow said tissue affecting device head to pass therethrough and therealong; and
a shifting mechanism configured for shifting said surgical device to an operative position within, or distal to, said hollow grasping head.

14. The system according to claim 13, wherein said surgical device includes a dissector head equipped with a set of first and second dissector jaws, and said surgical device includes a scissors head equipped with a set of first and second scissor jaws.

15. (canceled)

16. The system according to claim 13, wherein each of said first and second grasping jaws is configured as a half-tube with a toothed edge.

17. The system according to claim 13, wherein said tissue affecting device head is reversibly convertible between a tissue dissecting configuration and a tissue scissoring configuration.

18. The system according to claim 13, wherein said grasping head lumen is sized for housing said tissue affecting device head therein, thereby allowing combined and unified operation of said grasping head and said tissue affecting device head as an integral grasper head.

19. (canceled)

20. The system according to claim 13, wherein each of said first and second grasping jaws includes a sloped surface shaped and dimensioned such that said tissue affecting device head, when engaging and pushing against said sloped surface, forces said grasping jaws to radially retreat so as to allow said tissue tissue affecting device head to advance across distal end of said grasping head.

21. The system according to claim 13, wherein said grasping apparatus includes an outer tube configured for connecting between said tissue grasping manipulator and said grasping head.

22. The system according to claim 13, further comprising a cautery electrode in a form of a hook connected with cautery means, said cautery electrode is extendable from a recess in said grasping head and is configured for being forced to revolve from a horizontal position to a vertical position when extending from said recess to a fully deployed position.

23.-24. (canceled)

25. A method for performing surgical interventions in a body of a subject, the method comprising:
introducing, through an incision in said body, a surgical device comprising a tissue affecting device head extending from a distal end of a longitudinal shaft and having pivotally connected first and second dissector jaws, and further comprising a first scissors blade and a second scissors blade each sized and configured for sliding, and locking within a corresponding one of said first and second dissector jaws, wherein said tissue affecting device head is reversibly convertible between a tissue dissecting configuration and a tissue scissoring configuration, and between an opened state and a closed state via a control mechanism extending from proximal end of said longitudinal shaft;
dissecting tissue using said surgical device when said tissue affecting device head is in said tissue dissecting configuration, and, by opening said tissue affecting device head and closing said tissue affecting device head around said tissue;
converting said tissue affecting device head into said tissue scissoring configuration by said sliding and locking said first and second scissors blades within said first and second dissector jaws; and
cutting said tissue by opening said tissue affecting device head and closing said tissue affecting device head around said tissue.

26. The method according to claim 25, further comprising introducing a tissue grasping apparatus via said incision, wherein said tissue grasping apparatus includes an outer tube sized to accommodate and facilitate sliding therethrough said longitudinal shaft and said tissue affecting device head, wherein said outer tube is connected via a distal end thereof to a hollow grasping head having pivotally connected first and second grasping jaws, and said grasping head is forcibly transformable between an opened state and a closed state.

27. The method according to claim 26, comprising sliding said surgical device through said tissue grasping apparatus.

28. The method according to claim 26, comprising grasping said tissue by opening said grasping head and closing said grasping head around said tissue.

29.-32. (canceled)
